SEQUENCE LISTING (12 sequences)

SEQ ID NO: 1 - DNA, 20 bp, Artificial sequence
  Chemically synthesized
gcttccttgtatttgctggc

SEQ ID NO: 2 - DNA, 21 bp, Artificial sequence
  Chemically synthesized
ttctccacctcttcactgttg

SEQ ID NO: 3 - DNA, 25 bp, Artificial sequence
  Chemically synthesized
tggatggttctcagtcggtcactct

SEQ ID NO: 4 - DNA, 22 bp, Artificial sequence
  Chemically synthesized
tctcaatgaatctcaacgtccg

SEQ ID NO: 5 - DNA, 20 bp, Artificial sequence
  Chemically synthesized
atctgttctccacccctttg

SEQ ID NO: 6 - DNA, 24 bp, Artificial sequence
  Chemically synthesized
atcaacatgtcgtctgtggcctcc

SEQ ID NO: 7 - DNA, 20 bp, Artificial sequence
  Chemically synthesized
ggctgctgcaggaagtaaga

SEQ ID NO: 8 - DNA, 20 bp, Artificial sequence
  Chemically synthesized
atccctgccttctcaatggc

SEQ ID NO: 9 - DNA, 21 bp, Artificial sequence
  Chemically synthesized
caggtgaagatcgtggaagtc

SEQ ID NO: 10 - DNA, 20 bp, Artificial sequence
  Chemically synthesized
tgggagaaacaaagctggtg

SEQ ID NO: 11 - DNA, 24 bp, Artificial sequence
  Chemically synthesized
tcaacagcaactcccactcttcca

SEQ ID NO: 12 - DNA, 24 bp, Artificial sequence
  Chemically synthesized
accctgttgctgtagccgtattca

(19) United States
(12) Patent Application Publication
(10) Pub. No.: US 2018/0008629 A1
(43) Pub. Date: Jan. 11, 2018

(54) Compositions and Methods for Treating NLRP3 Inflammasome-Related Diseases and Disorders

(71) Applicant: Yale University, New Haven, CT (US)

(72) Inventor: Vishwa Deep Dixit, Woodbridge, CT (US)

(21) Appl. No.: 15/545,386

(22) PCT Filed: Jan. 27, 2016
(86) PCT No.: PCT/US16/15144

§ 371 Date: Jul. 21, 2017

Related U.S. Application Data
(60) Provisional application No. 62/109,586, filed on Jan. 29, 2015.
(60) Provisional application No. 62/190,852, filed on Jul. 10, 2015.

(51) Int. Cl. A61K 31/765; A61K 31/19; A61K 9/127; A61K 45/06

(52) U.S. Cl. CPC A61K 31/765; A61K 45/06; A61K 31/19; A61K 47/6911; A61K 47/6951; A61K 9/1271

(57) ABSTRACT

The present invention provides compositions and methods for treating or preventing an NLRP3 immunosome-related disorder. In one embodiment, the method includes administering a therapeutically effective amount of a composition comprising at least one NLRP3 inflammasome inhibitor to a subject in need thereof.

### CROSS REFERENCE TO RELATED APPLICATIONS

This application claims the benefit of priority to U.S. Provisional Patent Application Nos. 62/109,586, filed on Jan. 29, 2015, and 62/190,852, filed on Jul. 10, 2015, all of which are herein incorporated by reference in their entireties.

### STATEMENT REGARDING FEDERALLY SPONSORED RESEARCH OR DEVELOPMENT

This invention was made with government support under grant numbers AG043608, AG31797, DK090556 and AI105097 awarded by the National Institutes of Health. The government has certain rights in the invention.

### BACKGROUND OF THE INVENTION

NOD-like receptor family, pyrin domain containing 3 (NLRP3) inflammasome activation is implicated in causing diseases such as gout, atherosclerosis, type-2 diabetes, Alzheimers diseases, multiple sclerosis, silicosis and age-related bone loss. Ketone bodies, such as β-hydroxybutyrate (BHB) and acetoacetate, support mammalian survival during periods of starvation by serving as a source of ATP (Newman and Verdin, 2014, Trends Endocrinol. Metab. 25:42-52; Cotter et al., 2013, Am. J. Physiol. Heart Circ. Physiol. 304:H1060-1076). Prolonged fasting reduces inflammation as the innate immune system adapts to low glucose and energy metabolism switches towards mitochondrial fatty acid oxidation (Shido et al., 1989, J. Appl. Physiol. 67:963-969; Johnson et al., 2007, Free Radic. Biol. Med. 42:665-674; Mercken et al., 2013, Aging Cell 12:645-651). Consistent with this, inhibition of glycolysis in macrophages lowers pro-inflammatory cytokine IL-1β (McGettrick and O'Neill, 2013, J. Biol. Chem. 288:22893-22288).

Macrophage expressed NLRP3 inflammasome controls the activation of caspase-1 and release of pro-inflammatory cytokines IL-1β and IL-18 (Martinon et al., 2009, Annu. Rev. Immunol. 27:229; Lamkanfi and Dixit, 2014, Cell 157:1013-1022; Wen et al., 2013, Immunity 39:432-441; Latz et al., 2013, Nat. Rev. Immunol. 13:397-411; Franchi et al., 2009, Nat. Immunol. 10:241-247). NLRP3 inflammasome is an important innate immune sensor that can get activated in response to structurally diverse damage-associated molecular patterns (DAMPs), such as toxins (Lamkanfi and Dixit, 2014, Cell 157:1013-1022), ATP (Lamkanfi and Dixit, 2014, Cell 157:1013-1022), excess glucose (Martinon et al., 2009, Annu. Rev. Immunol. 27:229), ceramides (Vandanmagsar et al., 2011, Nat. Med. 17:179-188), amyloids (Masters et al., 2010, Nat. Immunol. 11:897-904; Heneka et al., 2013, Nature 493:674-678), urate (Martinon et al., 2006, Nature 440:237-241), and cholesterol crystals (Duewell et al., 2010, Nature 464:1357-1361). Ablation of NLRP3 lowers type 2 diabetes (Vandanmagsar et al., 2011, Nat. Med. 17:179-188, Heneka et al., 2013, Nature 493:674-678, Wen et al., 2011, Nat. Immunol. 12:408-415), atherosclerosis (Duewell et al., 2010, Nature 464:1357-1361), multiple sclerosis (Shaw et al., 2010, J. Immunol. 184:4610-4614), Alzheimer's disease (Heneka et al., 2013, Nature 493:674-678), age-related functional decline (Youm et al., 2013, Cell Metab. 18:519-532), bone loss (Youm et al., 2013, Cell Metab. 18:519-532), and gout (Martinon et al., 2006, Nature 440:237-241). In addition, therapies for patients with gain of function mutation of NLRP3 are not fully adequate in resolving chronic inflammation. Thus, identification of endogenous mechanisms that control NLRP3 inflammasome deactivation may provide insights in controlling several chronic diseases. Although it is known that immune-metabolic interactions via glycolytic inhibition dampen pro-inflammatory responses (McGettrick and O'Neill, 2013, J. Biol. Chem. 288:22893-22288), it is not known whether alternate metabolic fuels such as ketones that are produced during energy deficit impact the innate immune sensors. Currently, there are no effective therapies to lower NLRP3 inflammasome activity in clinic.

There is a need in the art for methods to reduce the activity of the NLRP3 inflammasome for the treatment of NLRP3-related diseases or disorders, such as gout or atherosclerosis. The present invention addresses this unmet need.

### BRIEF SUMMARY OF THE INVENTION

The present invention includes a method for treating or preventing an NLRP3 inflammasome-related disease or disorder. The method includes administering a therapeutically effective amount of a composition comprising at least one NLRP3 inflammasome inhibitor to a subject in need thereof. In another embodiment, the NLRP3 inflammasome inhibitor is a compound that is a ketone body. In one embodiment, the compound further comprises at least one hydroxyl group. In another embodiment, the compound is selected from the group consisting of β-hydroxybutyrate (BHB), γ-hydroxybutyrate (GHB), α-hydroxybutyrate (α-HB), polyhydroxybutyrate, a salt thereof, and any combinations thereof. In another embodiment, the compound is enantiomerically pure. In another embodiment, the compound is (S)-β-hydroxybutyrate [(S)-BHB]. In another embodiment, the compound is conjugated to a nanoparticle. In another embodiment, the nanoparticle is a nanolipogel. In another embodiment, the nanolipogel comprises at least one liposome and a core. In another embodiment, the liposome is comprised of cholesterol, at least one phosphatidylcholine lipid, and at least one PEG-lipid. In another embodiment, the at least one phosphatidylcholine lipid is L-α-phosphatidylcholine. In another embodiment, the PEG-lipid is distearoyl-phosphatidylethanolamine-N-[methoxy(polyethene glycol)-2000] (DSPE-PEG-2000). In another embodiment, the molar ratio of cholesterol to photphatidylcholine lipid to PEG-lipid is about 2:1:0.1 In another embodiment, the core comprises the at least one inhibitor, at least one host material, and at least one photoinitiator. In another embodiment, the at least one inhibitor is BHB. In another embodiment, the host material is at least one cyclodextrin. In another embodiment, the at least one cyclodextrin is selected from the group consisting of acrylate-β-cyclodextrin, hydroxypropyl-β-cyclodextrin, and mixtures thereof. In another embodiment, the at least one photoinitiator is an Irgacure photoinitiator. In another embodiment, the disease or disorder is selected from the group consisting of gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multisystem inflammatory disease (NOMID), Muckle-Wells Syndrome (MWS) and Familial Cold Autoinflammatory syndrome (FCAS). In another embodiment, the method further includes administering to the subject a ketogenic diet. In another embodiment, the subject is a human.

The present invention also includes a composition comprising at least one NLRP3 inflammasome inhibitor conjugated to a nanoparticle. In one embodiment, the nanoparticle is a nanolipogel. In another embodiment, the nanolipogel comprises at least one liposome and a core. In another embodiment, the liposome comprises cholesterol, at least one phosphatidylcholine lipid, and at least one PEG-lipid. In another embodiment, the at least one phosphatidylcholine lipid is L-α-phosphatidylcholine. In another embodiment, the PEG-lipid is distearoyl-phosphatidylethanolamine-N-[methoxy(polyethene glycol)-2000] (DSPE-PEG-2000). In another embodiment, the molar ratio of cholesterol to photphatidylcholine lipid to PEG-lipid is about 2:1:0.1 In another embodiment, the core is comprised of the at least one inhibitor, at least one host material, and at least one photoinitiator. In another embodiment, the at least one inhibitor is BHB. In another embodiment, the host material is at least one cyclodextrin. In another embodiment, the at least one cyclodextrin is selected from the group consisting of acrylate-β-cyclodextrin, hydroxypropyl-β-cyclodextrin, and mixtures thereof. In another embodiment, the at least one photoinitiator is an Irgacure photoinitiator. In another embodiment, the composition further includes at least one pharmaceutically acceptable carrier.

The present invention also includes a method for treating or preventing an NLRP3 inflammasome-related disease or disorder. The method includes administering a therapeutically effective amount of a composition comprising at least one NLRP3 inflammasome inhibitor to a joint in a subject in need thereof. In one embodiment, the NLRP3 inflammasome inhibitor is a compound that is a ketone body. In another embodiment, the compound further comprises at least one hydroxyl group. In another embodiment, the compound is selected from the group consisting of β-hydroxybutyrate (BHB), γ-hydroxybutyrate (GHB), α-hydroxybutyrate (α-HB), polyhydroxybutyrate, a salt thereof, and any combinations thereof. In another embodiment, the compound is enantiomerically pure. In another embodiment, the compound is (S)-β-hydroxybutyrate [(S)-BHB]. In another embodiment, the disease or disorder is selected from the group consisting of gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multisystem inflammatory disease (NOMID), Muckle-Wells Syndrome (MWS) and Familial Cold Autoinflammatory syndrome (FCAS). In another embodiment, the composition is administered by injection directly into the joint. In another embodiment, the composition is administered topically on or around the joint.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1K, depicts data demonstrating how β-hydroxybutyrate (BHB) specifically inhibits the NLRP3 inflammasome. FIG. 1A is a representative immunoblot analysis of caspase-1 (active subunit p20) and IL-1β (active p17) in the supernatant of BMDMs primed with LPS for 4 hours and stimulated with NLRP3 activator, ATP for 1 hour in presence of various concentration of D-BHB. The p20 caspase-1 band intensity was quantified by normalizing to inactive p48 procaspase-1. Each experiment was repeated with cells derived from more than 12 mice and repeated at least three times. FIG. 1B is a representative immunoblot analysis of caspace-1 activation in BMDMs stimulated with LPS and ATP and treated with BHB (10 mM), butyrate (10 mM), acetoacetate (10 mM) and acetate (10 mM). FIGS. 1C-1G are representative immunoblots demonstrating the effects of butyrate (FIG. 1C) and D-BHB concentrations on MSU induced caspase-1 activation in BMDMs. The LPS primed BMDMs were treated with BHB for 1 h in the presence of nigericin (10 μM) for 1 h (FIG. 1D), palmitate (200 μM) for 12 h (FIG. 1E), C6 ceramide for 6 h (80 μg/ml) (FIG. 1F), and sphingosine (50 μM) for 1 h (FIG. 1G). Caspase-1 cleavage was analysed by immunoblot. FIG. 1H is a representative immunoblot analysis of caspase-1 in BMDMs primed with TLR ligands lipid A, Pam3-CSK and LTA for 4 h and stimulated with ATP and increasing doses of BHB for 1 h. The immunoblot analysis of caspase-11 in BMDMs treated with LPS for 4 h and incubated with BHB for 1 h. FIGS. 1I-IJ are representative immunoblots of activation (p17 active form) and caspase-1 cleavage (p20) in BMDMs infected with F. tularensis (FIG. 1I) and S. typhimurium (FIG. 1J) and treated with different doses of BHB and IL-1β. All bar graphs represent quantitation of p20 caspase-1 band intensity as fold change by normalizing to inactive p48 procaspase-1. Each experiment was repeated with cells derived from 2 femurs of at least 4-6 mice and repeated three times. The data are presented as mean±SEM. *p<0.05. FIG. 1K is a representative immunoblot of active IL-1β (p17) analysed in supernatants by western blot.

FIG. 2, comprising FIGS. 2A-2B, depicts representative western blots of caspace-1 activation in BMDMs. FIG. 2A is a representative western blot analysis of caspase-1 (active subunit p20) in supernatant and cell lysates of BMDMs primed with LPS for 4 hours and stimulated with ATP for 1 hour in presence of various concentration of D-BHB. FIG. 2B is a representative western blot analysis of caspase-1 in BMDMs primed with LPS for 4 hours and stimulated with MSU or silica for 1 hour in the presence of various concentrations of D-BHB and butyrate (10 mM).

FIG. 3, comprising FIGS. 3A-3H, depicts data demonstrating that the BHB inhibits NLRP3 inflammasome independently of Gpr109a receptor and starvation regulated mechanisms. FIG. 3A is a representative immunoblot of caspase-1 activation in BMDMs primed with LPS and treated with rotenone (10 μM), ATP (5 μM) together with BHB (10 mM). FIG. 3B is a representative immunoblot of caspase-1 activation in LPS primed BMDMs cultured with ATP and BHB (10 mM) from control Atg5^{fl/fl} animals and mice lacking Atg5 in myeloid lineage. Caspase-1 was measured in cell lysates. FIG. 3C is a representative immunoblot of caspase-1 activation in BMDMs primed with LPS, pretreated with 3MA and epoxomicin for 30 min, and stimulated in the presence of ATP and BHB. FIG. 3D is a representative immunoblot of caspase-1 activation by western blot in LPS primed BMDM stimulated with ATP and BHB (10 mM) in the presence of an AMPK activator (AICAR, 2 mM) and an AMPK antagonist Compound C (25 μM). FIG. 3E is a bar graph showing proliferation of BMDMs in response to increasing concentration of BHB. FIGS. 3F-3G are representative immunoblots analyzing IL-1β activation in BMDMs from control and Gpr109a deficient mice activated with LPS and ATP and co-incubated with TSA (50 nM) and niacin (1 mM; FIG. 3F) or butyrate (10 mM) and acetoacetate (10 mM; FIG. 3G) and BHB (10, 20 mM). Caspase-1 cleavage was measured by western blot. FIG. 3H is a representative immunoblot of caspase-1 activation in BMDMs of WT and Gpr109a^{-/-} mice stimulated with LPS and ATP together with BHB enantiomer (S)-BHB. Each experiment was repeated with cells derived from 2 femurs of 3-6 mice and repeated twice. See FIG. 4A for the quantitation of p20 caspase1 band intensity from each experiment.

FIG. 4, comprising FIGS. 4A-4C, depicts data examining the mechanism of BHB's anti-inflammasome effects. FIG. 4A is a series of bar graphs depicting the quantitation of p20 caspase-1 and p17 band intensity as fold change by normalizing to inactive p48 procaspase-1 and proIL1β. Each experiment was repeated with cells derived from 2 femurs of at least 4-6 mice and repeated three times. The data are presented as mean±SEM. *p<0.05. FIG. 4B is a representative western blot analysis of caspase-1 in BMDMs primed with LPS for 4 hours and stimulated with hydrogen peroxide alone and with ATP for 1 hour. FIG. 4C is a western blot of caspase-1 in LPS primed BMDM stimulated with ATP and BHB (10 mM) in the presence of glycolytic inhibitor 2-deoxyglucose (1 mM), AMPK activator (AICAR, 2 mM), and AMPK antagonist Compound C (25 μM).

FIG. 5, comprising FIGS. 5A-5B, depicts data from experiments examining the mechanism of BHB's anti-inflammasome effects. FIG. 5A is a representative western blot analysis of caspase-1 in BMDMs primed with LPS for 4 hours and stimulated with 2DG, AICAR, Compound C and BHB for 1 h on indicated combinations. FIG. 5B is a representative western blot examining H3 acetylation in LPS primed BMDMs treated with ATP and S- or D-BHB (R-BHB) for 1 h.

FIG. 6, comprising FIGS. 6A-6F, depicts FIG. 6A is a schematic of the ketolytic pathway of generation of acetyl coA. FIG. 6B is a graph of the differential mRNA expression of ketolytic and ketogenic enzymes in M1 and M2 polarized macrophages. FIG. 6C is a western blot analysis of SCOT and HMGCL expression in BMDMs in response to inflammasome activators and butyrate and BHB. FIG. 6D is a representative immunoblot of the caspase-1 activation in LPS primed BMDMs incubated in the presence of TCA cycle entry inhibitor AOA (1 mM) for 1 hour in presence or absence of ATP and BHB. FIG. 6E is a representative immunoblot of the effect of enantiomer, (S)-BHB (L-BHB) on Nlrp3 induced caspase-1 activation in BMDMs. FIG. 6F is a series of bar graphs representing quantitation of p20 caspase1, p17, and SCOT band intensity as fold change by normalizing to inactive p48 procaspase-1, proIL-1β, and actin. Each experiment was repeated with cells derived from 2 femurs of at least 4-6 mice and repeated three times. The data are presented as mean±SEM. *p<0.05.

FIG. 7, comprising FIGS. 7A-7I, depicts experimental data demonstrating how BHB inhibits NLRP3 ligand dependent ASC oligomerization without undergoing mitochondrial oxidation. FIG. 7A is a representative western blot measuring SCOT in LPS primed BMDMs cultured with ATP and BHB (10, 20 mM) and acetoacetate (10 mM) from control and LysM::Cre Oxct^{fl/fl} mice lacking SCOT in myeloid lineage and caspase-1 cleavage. FIG. 7B is a representative immunoblot analysing caspase-1 in LPS and ATP stimulated BMDMs treated with BHB (10 mM), Sirt2 antagonist AGK2 (10 μM) and NAD+ (10 μM). FIGS. 7C-7D are representative immunoblots examining caspace-1 activation in WT or Sirt^{-/-} (FIG. 7C) or Ucp2^{-/-} (FIG. 7D) BMDMs stimulated with LPS and ATP and treated with BHB (10 mM) for 1 h. FIG. 7E is a graph depicting intracellular potassium levels in BMDMs stimulated with LPS and ATP in the presence of BHB (10 mM) measured using Inductively Coupled Mass Spectrometry (ICP-MS). FIGS. 7F-7G are bar graphs depicting the intracellular potassium levels in LPS primed BMDMs treated with ATP (FIG. 7F) or MSU (FIG. 7G) and BHB for 1 h measured using APG-1 dye that selectively binds potassium with an excitation emission spectra of 488-540 nm. FIG. 7H is a representative immunoblot examining disuccinimidyl suberate (DSS) cross-linked ASC in Nonidet P-40-insoluble pellet of BMDMs that were primed with LPS (4 h) and stimulated with ATP and BHB for 1 h. The bar graphs represent the quantification of band intensity of ASC dimer. The experiment was repeated three times with cells derived from 2 femurs of 4-6 mice. The data are presented as mean±SEM. *p<0.05. FIG. 7I is a series of images of BMDMs stimulated with LPS+ATP in the presence of BHB (10 mM) and stained using anti-ASC primary antibody and anti-rabbit Alexa fluor 488 conjugated secondary antibody for 1 h. The ASC specks (as arrow heads) were quantified using ImageJ software. At least 5 distinct fields were analyzed and a minimum of 550 cells from each treatment condition were quantified. The data are presented as mean±SEM. *p<0.05. See FIG. 6F for the quantitation of p20 caspase1 band intensity from each experiment.

FIG. 8, comprising FIGS. 8A-8G, depicts data demonstrating the effect of BHB on BMDMs. FIG. 8A is a bar graph of the intracellular potassium levels in LPS primed BMDMs treated with C6 ceramide and BHB for 1 h. FIG. 8B is a bar graph of the TNFα concentration of human monocytes stimulated with vehicle or LPS (1 μg/mL) for 4 h in the presence of increasing concentrations of BHB. The TNFα was measured in supernatants using ELISA. FIG. 8C is a bar graph of the quantitation of peritoneal cells stained with CD45 and Gr1 from mice treated intraperitoneally with MSU (3 mg) BHB-complexed nLGs (5 mg/kg b.w). (n=6/group). FIGS. 8D-8E depict data of peritoneal cells stained with Ly6C and Ly6G. FIG. 8D is a series of flow cytometry spectra showing nanolipogel, nanolipogel+MSU, and MSU+BHB nanolipogel treated peritoneal cells. FIG. 8E is a bar graph of Ly6C and Ly6G concentration in the cells. FIG. 8F is a graph of the data showing the number or cells migrated from double positive neutrophil infiltration in mice 4 h after treatment with MSU (2.5 mg) and BHB-nLGs (3 mg/kg b.w). For FIGS. 8D-8F, n=4/group. FIG. 8G is a series of bar graphs representing quantitation of p20 caspase1 and p17 band intensity as fold change by normalizing to inactive p48 procaspase-1 and proIL-1β. Each experiment was repeated with cells derived from 2 femurs of at least 4-6 mice and repeated three times. The data are presented as mean±SEM. *p<0.05.

FIG. 9, comprising FIGS. 9A-9L, depicts data demonstrating how BHB deactivates inflammasome in human monocytes and in mouse models of NLRP3 driven diseases. FIG. 9A-9B are a series of graphs depicting the concentration of IL-1β (FIG. 9A) and IL-18 (FIG. 9B) in human monocytes stimulated with vehicle or LPS (1 μg/mL) for 4 h in presence of increasing concentrations of BHB. The IL-1β and IL-18 concentrations were measured in supernatants using ELISA. The data are presented as mean±SEM. *p<0.05 (n=6). FIG. 9C is a representative immunoblot of the caspase-1 activation in BMDMs treated with ATP and different concentration of BHB-complexed nanolipogels (nLGs). FIG. 9D is a series of representative FACS dot plots of peritoneal cells stained with CD45 and Gr1 from mice treated intraperitoneally with MSU (3 mg) and/or BHB-nLGs (5 mg/kg b.w) (n=6/group). FIG. 9E is a graph of the concentration of IL-1β secretion from peritoneal cells cultured overnight. FIG. 9F is a graph of the serum IL-1β levels from mice challenged with MSU and treated with BHB-nLGs (5 mg/kg b.w). FIG. 9G is a representative immunoblot of BM cells from mice harboring the MWS mutation NLRP3A350V treated with 4-hydroxy tamoxifen on day 6 of macrophage differentiation in order to induce Cre recombination and activation of NLRP3. The LPS stimulated constitutively activated BMDMs harboring the NLRP3 gain of function mutation were treated with BHB-nLG and active IL-1β p17 in supernatants and caspase-1 activation was analyzed in cell lysates. FIGS. 9H-9I depict immunoblots from BM cells from mice harboring the FCAS mutation (NLRP3^{L351P}) treated with 4-hydroxy tamoxifen on day 6 of macrophage differentiation used to induce Cre recombination and constitutive NLRP3 activation. The western blot analysis of caspase-1 activation (FIG. 9H) and IL-1β (FIG. 9I) in BMDMs were primed with LPS alone (4 h) and treated with D-BHB (FIG. 9I) for 1 hour and various doses of D-BHB-nLGs. FIG. 9J is a representative immunoblot of ASC oligomerization (dimer and monomer formation) in DSS cross-linked ASC in NP40-insoluble pellet BMDMs from FCAS mice (n=6) treated with LPS and various concentrations of BHB-nLG analysed by western blot. See FIG. 8G for the quantitation of p20 caspase1, IL-1β p17 and ASC dimer band intensities from each experiment. FIG. 9K is a graph of neutrophil infiltration in NLRP3^{L351P} FCAS gain of function mice fed ketone diester diet (1,3-butanediol) for one week. Neutrophil infiltration in peritoneum was evaluated 3 days post tamoxifen Cre-induced NLRP3 mutation activation (n=6-8/group). The data are presented as mean±SEM. *p<0.05. FIG. 9L is an illustration of the mechanism of BHB-mediated immune-metabolic crosstalk that integrates negative energy balance to innate immune function by inhibition of the NLRP3 inflammasome in macrophages.

FIG. 10, comprising FIGS. 10A-10E, depicts data demonstrating the effect of BHB on BMDMs. FIG. 10A is a representative immunoblot of a western blot analysis of IL-1β in BMDMs primed with LPS alone and treated with or without tamoxifen. The BM cells from mice harbouring the FCAS mutation (NLRP3^{L351P}) were treated with tamoxifen on day 6 of macrophage differentiation to induce Cre recombination and activation of NLRP3. FIG. 10B is a representative immunoblot of IL-1β in BMDMs treated with D-BHB for 1 hour and various doses of D-BHB-nLGs. FIG. 10C is a representative immunoblot of IL-1β activation (p17) in BMDMs of FCAS mice (NLRP3^{L351P}) treated with LPS and D-BHB-nLGs at various concentrations. Cre was induced by tamoxifen injection 24 h before LPS treatment. FIG. 10D is a graph of blood glucose levels evaluated 3 days post tamoxifen Cre-induced NLRP3 mutation activation (n=6-8/group). FIG. 10E is a graph of the concentration of stained peritoneal cells in FCAS mice (NLRP3^{L351P}) treated with a ketone ester diet. The mice were fed chow and ketone ester diet (1,3 butanediol, 20% by volume) for one week and peritoneal cells were removed three days after tamoxifen-induced Cre recombination. The peritoneal cells were stained with CD11b and F4/80 and quantified by Flow Cytometry.

FIG. 11, comprising FIGS. 11A-11D, depicts data demonstrating the effect of a ketone diester diet in mice harbouring the FCAS mutation (NLRP3^{L351P}). The mice were treated with tamoxifen on day 6 of macrophage differentiation to induce Cre recombination and activation. The ketone diester did not impact the overall frequency of neutrophil (FIGS. 11A and 11B), macrophage (FIG. 11C), or T cell (FIG. 11D), numbers in the spleen.

FIG. 12 depicts western blot data demonstrating that 10 mM BHB blocks IL-1β production after NLRP3 activation in neutrophils. Neutrophils from young (3 month) and aged (24 months) mice were stimulated with LPS, and the NLRP3 inflammasome was activated by treatment with ATP and C6 Ceramide. BHB was provided at the same time of ATP and ceramide treatment. The western blot analysis shows that IL-1B activation (determined by the presence of the p17 active form) in response to NLRP3 activators is blocked by BHB in primary neutrophils.

FIG. 13, comprising FIGS. 13A-13C, depicts experimental data demonstrating that IL-1β secretion in adult and old neutrophils is NLRP3-dependent. Neutrophils from the femurs of adult and old mice were purified and analyzed for NLRP3 inflammasome components and activation. FIG. 13A is a series of images of Western blots measuring NLRP3, ASC, and β-Actin expression in unstimulated neutrophil cell lysates from adult and old mice. FIG. 13B is an image of a Western blot analyzing supernatants from adult and old neutrophils stimulated with LPS±ATP for IL-β secretion. FIG. 13C is a series of graphs demonstrating data of neutrophils from adult and old mice of the indicated genotype stimulated with LPS+ATP. IL-1β and TNFα were measured in the supernatants by Luminex. In FIGS. 13A and 13B, blots are representative of at least 3 independent experiments. Each adult and old sample is pooled from n=4-6 mice. In FIG. 13C, data is combined from two independent experiments. Each dot represents an individual mouse. *p<0.05. Statistical differences were calculated by 1-way ANOVA with Bonferroni's post test for multiple comparisons.

FIG. 14, comprising FIGS. 14A-14D, depicts experimental data demonstrating that neutrophils contain ketone metabolism machinery and BHB inhibits NLRP3 inflammasome activation. Adult and old neutrophils were analyzed for ketogenic and ketolytic enzyme expression. FIG. 14A is a series of graphs of experimental data of gene expression measured by RT-PCR. Expression was normalized to Gapdh expression and is represented as expression relative to adult gene expression. FIG. 14B is an image of a Western blot of SCOT, HMGCL, and β-Actin protein expression assessed in unstimulated adult and old neutrophils. FIG. 14C is an image of a Western blot of dose response to BHB measured by assaying IL-1β secretion in supernatants from adult neutrophils. FIG. 14D is an image of a Western blot of IL-1β in supernatants from adult and old neutrophils stimulated with LPS+ATP or LPS+ceramide. In FIG. 14A, each dot represents a pooled sample of n=2 mice. In FIGS. 14B-14D, each blot is representative of at least two independent experiments. Each sample analyzed by Western blot is pooled from n=4-6 mice per experiment.

FIG. 15, comprising FIGS. 15A-15E, depicts experimental data demonstrating that inhibitory effects of BHB involve physically blocking inflammasome assembly. Adult neutrophils were stimulated with LPS+ATP+BHB to test the mechanism by which BHB blocks IL-1β secretion. FIG. 15A is an image of a Western blot of LPS-primed neutrophils stimulated with ATP in the presence of BHB or niacin. Supernatants were analyzed for IL-1β secretion by Western blot. FIG. 15B is an image of a Western blot of wildtype of mCAT neutrophils stimulated with LPS+ATP+BHB. Supernatants were analyzed for IL-1β secretion by Western blot. Catalase and β-Actin expression were measured by Western blot in neutrophil cell lysates. FIG. 15C is an image of a Western blot of LPS-primed neutrophils stimulated with ATP+BHB in the presence of 3-MA or AOA as indicated. Supernatants were analyzed for IL-1β secretion by Western blot. FIG. 15D is an image of a Western blot of the enantiomer S-BHB tested in a dose response for the ability to inhibit inflammasome activation. Supernatants were analyzed for IL-1β secretion by Western blot. FIG. 15E is an image of a Western blot of FCAS neutrophils incubated with 4-OHT followed by LPS priming. BHB nanolipogels were added to test inhibition of inflammasome activation. Supernatants were analyzed for IL-1β secretion by Western blot. For all blots, each sample is pooled from at least n=4 mice per experiment. Each blot is representative of at least two independent experiments.

FIG. 16, comprising FIGS. 16A-16C, depicts experimental data demonstrating that a ketogenic diet prevents neutrophil hyperactivation in a peritonitis model in old mice. Old mice were fed a ketogenic diet for one week prior to MSU challenge to induce neutrophil infiltration and inflammasome activation. FIG. 16A is a series of graphs of experimental data of body weights and blood BHB concentrations measured daily. FIG. 16B is a series of graphs of experimental data from four hours after MSU injection. Total peritoneal cells were collected and analyzed by FACS to enumerate total neutrophil infiltration. FIG. 16C is a series of graphs of experimental data of gene expression within peritoneal cells measured by RT-PCR. Expression was normalized to Gapdh expression and data are represented as expression relative to adults. In FIGS. 16B and 16C, data were pooled from two independent experiments. Statistical differences were calculated by 2-way ANOVA (FIG. 16A) or 1-way ANOVA (FIG. 16B). Each dot represents an individual mouse. *p<0.05, ****p<0.0001.

FIG. 17, comprising FIGS. 17A-17B, depicts experimental data demonstrating neutrophil identification and enumeration. Adult and old bone marrow was harvested from femurs and analyzed for neutrophils. FIG. 17A is a series of images of a representative gating strategy to identify neutrophils from by multi-color flow cytometry. FIG. 17B is a series of graphs of experimental data demonstrating the enumeration of total cells after RBC lysis and calculation of total neutrophils. Data are representative of 2 independent experiments, each containing 8 mice per group. Statistical differences were calculated by unpaired student's t-test. Each dot represents an individual mouse. **p<0.01.

FIG. 18 depicts experimental data demonstrating the purity of adult and old neutrophils after magnetic enrichment. Adult and old neutrophils were enriched from bone marrow for all ex vivo stimulation experiments. Representative flow cytometry analysis show comparable purity between adult and old samples.

FIG. 19, comprising FIGS. 19A-19F, depicts experimental data demonstrating that BHB does not increase infection severity but still prevents neutrophil hyperactivation during peritonitis. Mice were fed a ketogenic diet for 1 week prior to increase BHB levels and then infection or peritonitis was induced by injection of monosodium urate. FIG. 19A is a graph of experimental data depicting body weights and blood BHB concentrations were measured daily in old mice during ketogenic diet feeding, prior to MSU injection. FIG. 19B is a graph of experimental data depicting pro-inflammatory cytokine Il1b, Nlrp3 and Tnf gene expression measured by RT-PCR in isolated peritoneal cells of old mice fed chow and ketogenic diet that were treated with i.p urate injections to induce periitonitis. Expression was normalized to Gapdh expression and data are represented as expression relative to sham Old mice. FIG. 19C is a graph of experimental data depicting blood BHB levels measured in adult mice 24 hr post-infection with Staphylococcus aureus. FIG. 19D is a graph of experimental data depicting quantified total cells collected from brocho-aleveolar lavage BAL fluid from lungs 24 hr post-infection. FIG. 19E is a graph of experimental data depicting bacterial burdens in lung tissue determined 24 hr after infection. FIG. 19F is a graph of experimental data depicting body weights measured daily for 7 days following S. aureus infection until all mice returned to baseline body weight. All data are pooled from at least two independent experiments. Statistical differences were calculated by ANOVA or unpaired student's t-test. Each dot represents an individual mouse. *p<0.05, ****p<0.0001.

FIG. 20 is a graph of experimental data demonstrating that BHB inhibits IL1B production from human neutrophils irrespective of age. Peripheral blood neutrophils from adult (30-40 years) and old (65-75 years) were enriched and stimulated as indicated. IL-1β secretion was measured in culture supernatants by ELISA. Data are expressed as mean±S.E.M (*p<0.01).

FIG. 21, comprising FIGS. 21A-21D, depicts experimental data demonstrating that increasing the levels of BHB protects against Gout-induced inflammation in rats. Rats were fed a ketogenic diet for 1 week prior to induction of gout by intra-articular injection of MSU. FIG. 19A is a graph of experimental data depicting blood BHB levels measured in rats after 1 week ketogenic diet feeding, prior to injection with MSU. FIG. 19B is a graph of experimental data depicting knee thickness measured daily. FIG. 19C is a graph of experimental data depicting the increase in knee swelling relative to baseline thickness 2 days after gout induction. FIG. 19D is a graph of experimental data depicting serum IL-1β measured by ELISA on day 2 post-MSU injection

### DETAILED DESCRIPTION

The present invention is based on the discovery that inhibition of the NLRP3 inflammasome by β-hydroxybutyrate (BHB) is useful for the treatment of diseases and disorders associated with the NLRP3 inflammasome, such as chronic inflammatory diseases. Thus, the present invention includes a method for treating or preventing an NLRP3 inflammasome-related disease or disorder comprising administering a therapeutically effective amount of a composition comprising an NLRP3 inflammasome inhibitor to a subject in need thereof. In one embodiment, the method of the invention includes a method of treating or preventing an NLRP3 inflammasome-related disease selected from the group consisting of gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multisystem inflammatory disease (NOMID), Muckle-Wells Syndrome (MWS) and Familial Cold Autoinflammatory syndrome (FCAS).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered.”

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or disorder as determined by any means suitable in the art.

As used herein, the term “pharmaceutical composition” to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs or symptoms of disease or disorder, for the purpose of diminishing or eliminating those signs or symptoms.

As used herein, “treating a disease or disorder” means reducing the severity and/or frequency with which a sign or symptom of a disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or disorder associated with NLRP3 inflammasome activation, including alleviating signs and/or symptoms of such diseases or disorders.

As used herein, the term “stereoisomer” refers to compounds that have their atoms connected in the same order but differ in the arrangement of their atoms in space. (e.g., L-alanine and D-alanine).

As used herein, the terms “(S)-BHB” and “L-BHB” are interchangeable and refer to (S)-3-hydroxybutyric acid.

As used herein, the terms “(R)-BHB” and “D-BHB” are interchangeable and refer to (R)-3-hydroxybutyric acid.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

As used herein, the term “liposome” refers to a microscopic, fluid-filled structure, with walls comprising one or more layers of phospholipids and molecules similar in physical and/or chemical properties to those that make up mammalian cell membranes, such as, but not limited to, cholesterol, stearylamine, or phosphatidylcholine. Liposomes can be formulated to incorporate a wide range of materials as a payload either in the aqueous or in the lipid compartments.

The term “phospholipids” refers to any member of a large class of fatlike organic compounds that in their molecular structure resemble the triglycerides, except for the replacement of a fatty acid with a phosphate-containing polar group. One end of the molecule is soluble in water (hydrophilic) and water solutions. The other, fatty acid, end is soluble in fats (hydrophobic). In watery environments, phospholipids naturally combine to form a two-layer structure (lipid bilayer) with the fat-soluble ends sandwiched in the middle and the water-soluble ends sticking out. Such lipid bilayers are the structural basis of cell membranes and liposomes.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based in part on the discovery that (3-hydroxybutyrate (BHB) inhibits NLRP3 inflammasome activity in macrophages and neutrophils in response to diverse NLRP3 pro-inflammatory inducers without deactivating the NLRC4, AIM2, or non-canonical caspase-11 inflammasomes. The present invention provides compositions and methods that are useful in inhibiting the activity of the NLRP3 inflammasome in a mammal, such as a human. In particular, the present invention provides compositions and methods that are useful for inhibiting the NLRP3 inflammasome for the treatment of NLRP3 inflammasome-related diseases and disorders, such as gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multisystem inflammatory disease (NOMID), Muckle-Wells Syndrome (MWS) and Familial Cold Autoinflammatory syndrome (FCAS), in a mammal.

The present invention is related to the discovery that BHB's inhibitory effects on the NLRP3 inflammasome activation are not dependent on classical starvation regulated mechanisms like 5′ adenosine monophosphate-activated protein kinase (AMPK), reactive oxygen species (ROS), autophagy or glycolytic inhibition. Furthermore, it was found that BHB blocked NLRP3 independently of mitochondrial uncoupling or oxidation for energetic purposes, without requirement for GPR109a or histone acetylation. BHB was also found to deactivate the NLRP3 inflammasome in human monocytes and in mouse models of urate induced inflammation and NLRP3-related autoinflammatory diseases such as Muckle-Wells Syndrome (MWS) and Familial Cold Autoinflammatory syndrome (FCAS). Accordingly, the invention provides compositions and methods for treating NLRP3-related diseases and disorders by inhibiting the NLRP3 inflammasome using a compound that targets NLRP3 inflammasome activity.

In one embodiment, the invention comprise administering a composition comprising an NLRP3 inflammasome inhibitor to a mammal exhibiting increased levels of NLRP3 inflammasome activity or determined to be at risk for developing increased levels of NLRP3 inflammasome activity. The methods of the present invention further comprise administering a composition comprising an NLRP3 inflammasome inhibitor to a mammal that has been diagnosed with an NLRP3 inflammasome-related disease or disorder, or who has symptoms or signs of an NLRP3 inflammasome-related disease or disorder.

The invention may be practiced in any subject diagnosed with, or at risk of developing an NLRP3 inflammasome-related disease or disorder. In some embodiments, the subject is a mammal. In some embodiments, the subject is a human.

Inhibiting NLRP3 inflammasome activity may be accomplished using any method known to the skilled artisan. Examples of methods to inhibit NLRP3 inflammasome activity include, but are not limited to, directly blocking the assembly of the NLRP3 inflammasome complex by inhibiting the oligomerization of inflammasome adaptor protein ASC (also called PYCARD (PYD and CARD domain containing)), decreasing expression of an endogenous NLRP3 inflammasome gene, decreasing expression of NLRP3 inflammasome mRNA, and inhibiting activity of NLRP3 inflammasome protein. Decreasing expression of an endogenous NLRP3 inflammasome gene includes providing a specific inhibitor of NLRP3 inflammasome gene expression. Decreasing expression of NLRP3 inflammasome mRNA or NLRP3 inflammasome protein includes decreasing the half-life or stability of NLRP3 inflammasome mRNA or decreasing expression of NLRP3 inflammasome mRNA. An NLRP3 inflammasome inhibitor may therefore be a compound or composition that decreases expression of an NLRP3 inflammasome gene, a compound or composition that decreases NLRP3 inflammasome mRNA half-life, stability and/or expression, or a compound or composition that inhibits NLRP3 inflammasome protein function. Examples of an NLRP3 inflammasome inhibitor include, but are not limited to, any type of compound, including a polypeptide, a peptide, a peptidomemetic, a nucleic acid, an siRNA, a microRNA, an antisense nucleic acid, an aptamer, a small molecule, an antibody, a ribozyme, an expression vector encoding a transdominant negative mutant, and combinations thereof. In one embodiment, the inhibitory effect of a therapeutic agent on NLRP3 inflammasome expression, function, or activity is indirect. In one embodiment, the present invention provides a method comprising administering a NLRP3 inflammasome inhibitor known in the art or discovered in the future.

### Compositions

### Compounds

In one embodiment, the NLRP3 inflammasome inhibitor is a compound. Any compound that inhibits the activity of the NLRP3 inflammasome is contemplated for use as an NLRP3 inflammasome inhibitor of the invention. In one embodiment, the inhibitor prevents potassium ion (K⁺) efflux from macrophages in response to NLRP3 inflammasome activators, thereby blocking the activation of caspase-1 and proinflammatory cytokine IL-1β and IL-18. In another embodiment, the inhibitor prevents oligomerization of the inflammasome adaptor protein ASC in response to NLRP3 inflammasome activators. ASC protein oligomerization is critical for assembly of the functional NLRP3 inflammasome complex. In another embodiment, the inhibitor inhibits NLRP3 inflammasome activity in neutrophils. Like macrophages, neutrophils also produce IL-1β and IL-18, resulting in inflammation.

In one embodiment, the compound is a ketone body. In one embodiment, the compound is β-hydroxybutyrate (BHB). BHB is a vital source of ATP during neonatal period, fasting, starvation, exercise or when there is reduced availability of glucose or carbohydrates as fuel. As demonstrated herein, BHB was found to inhibit NLRP3 inflammasome activation in macrophages in response to a wide variety of disease inducers. In another embodiment, the compound further comprises at least one hydroxyl (—OH) group. Although not wishing to be bound by any particular theory, the results demonstrated herein suggest that the presence of the β-hydroxyl group on BHB is critical for inhibition of NLRP3 inflammasome activity. Accordingly, in one embodiment, a compound of the present invention is a ketone body comprising at least one hydroxyl group. In one embodiment, the compound is γ-hydroxybutyrate (GHB). In another embodiment, the compound is polyhydroxylbutyrate. In another embodiment, the compound is α-hydroxybutyrate (α-HB).

The compounds of the invention may possess one or more stereocenters, and each stereocenter may exist independently in either the R or S configuration. As would be understood by one of ordinary skill in the art, the compound may be stereoisomers in either the D or L configuration. The results described herein demonstrate that the S enantiomer of BHB [(S)-BHB] efficiently inhibited NLRP3 inflammasome activity. Although not wishing to be bound by any particular theory, the results described elsewhere herein suggest that (S)-BHB is biologically inert because it does not enter the tricarboxylic acid cycle (TCA cycle) and is therefore not oxidized, resulting in a longer half-life in vivo. In one embodiment, the compound of the invention is (S)-BHB.

The present invention therefore includes any possible stereoisomers, enantiomers, diastereomers, racemates, salts, or mixtures thereof of the compounds of the invention that are efficacious in the treatment of an NLRP3 inflammasome-related disease or disorder. The isomers resulting from the presence of a chiral center comprise a pair of non-superimposable isomers that are called “enantiomers.” Single enantiomers of a pure compound are optically active, i.e., they are capable of rotating the plane of plane polarized light. The present invention is meant to encompass diastereoisomers as well as their racemic and resolved, diastereomerically and enantiomerically pure forms and salts thereof. As used herein, the terms “enantiomerically pure form” or “enantiomerically pure” refer to a compound that has been substantially purified from the corresponding optical isomer(s) of the same formula. Preferably, the compound is at least about 80% pure, at least about 90% pure, at least 98% pure, or at least about 99% pure, by weight.

In one embodiment, compounds described herein are present in optically active or racemic forms. In another embodiment, the compound of the invention is the S enantiomer. In one embodiment, the compound of the invention is the R enantiomer. In another embodiment, the compound of the invention is the D enantiomer. In another embodiment, the compound of the invention is the L enantiomer. It is to be understood that the compounds described herein encompass racemic, optically-active, regioisomeric and stereoisomeric forms, or combinations thereof that possess the therapeutically useful properties described herein. In one embodiment, a mixture of one or more isomer is utilized as the therapeutic compound described herein. In another embodiment, compounds described herein contain one or more chiral centers.

### Nanoparticles

In another aspect of the invention, the inhibitor is conjugated to a nanoparticle. Any nanoparticle which may improve the biological properties of the inhibitor is contemplated for use within the invention. In one embodiment, the nanoparticle is a nanolipogel (nLG). “Nanolipogel,” as used herein, refers to a core-shell nanoparticle having a polymer matrix core, which can contain a host molecule, within a liposomal shell, which may be unilamellar or bilamellar, and optionally crosslinked. Nanolipogels are core-shell nanoparticulates that combine the advantages of both liposomes and polymer-based particles for sustained delivery of active agents.

In one embodiment, the nanolipogel comprises at least one liposome. The liposomes may be prepared according to any method known in the art. In one embodiment, the liposomes are prepared by placing a mixture of a solution comprising at least one lipid under a stream of gas, such as nitrogen gas, in order to evaporate off the solvent of the solution, and then lyophilizing the mixture after extrusion to produce the liposomes. The lyophilized liposomes can then be rehydrated in order to incorporate additional agents, such as the inhibitors of the invention, in order to form nanolipogels.

In some embodiments, the liposomes of the present invention comprise one or materials that form a lipid bilayer. In one embodiment, the liposomes of the present invention comprise cholesterol. As used herein, the term “cholesterol” refers to 2,15-dimethyl-14-(1,5-dimethylhexyl)tetracyclo[8.7.0.0^{2,7}.0^{11.15}]heptadec-7-en-5-ol. In one embodiment, the liposomes comprise about 25-45 mol percent of cholesterol. In another embodiment, the liposomes contain about 30-35 mol percent cholesterol. In another embodiment, the liposomes contain about 33 mol percent cholesterol.

The liposomes may also comprise at least one lipid. The liposome can contain, for example, two, three, four, five, six, or seven or more lipids. In one embodiment, the lipid comprises two lipids. In one embodiment, the lipid is a phosphatidylcholine lipid. As used herein, the term “phosphatidylcholine lipid” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6-24 carbon atoms. Phosphatidylcholine lipids can include synthetic and naturally-derived 1,2-diacyl-sn-glycero-3-phosphocholines. Non-limiting examples of phosphatidylcholine lipids include L-α-phosphatidylcholine (1,2-diacyl-sn-glycero-3-phosphocholine), 1,2-distearoyl-sn-glycero-3 phosphocholine (distearoylphosphatidylcholine; DSPC), 1,2-dipalmitoyl-sn-glycero-3 phosphocholine (dipalmitoylphosphatidylcholine; DPPC), 1-myristoyl-2-palmitoyl-sn-glycero-3 phosphocholine (MPPC), 1-palmitoyl-2-myristoyl-sn-glycero-3 phosphocholine (PMPC), 1-myristoyl-2-stearoyl-sn-glycero-3 phosphocholine (MSPC), 1-palmitoyl-2-stearoyl-sn-glycero-3 phosphocholine (PSPC), 1-stearoyl-2-palmitoyl-sn-glycero-3 phosphocholine (SPPC), and 1-stearoyl-2-myristoyl-sn-glycero-3 phosphocholine (SMPC). In one embodiment, the phosphatidylcholine lipid is L-α-phosphatidylcholine.

Examples of unsaturated phosphatidylcholine lipids include, but are not limited to, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (palmitoyloleoylphosphatidylcholine; SOPC), 1-stearoyl-2-linoleoyl-sn-glycero-3-phosphocholine, 1-oleoyl-2-myristoyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (OSPC). Other lipid extracts, such as egg phosphatidylcholine lipid, heart extract, brain extract, liver extract, soy phosphatidylcholine lipid, and hydrogenated soy phosphatidylcholine lipid (HSPC) may also be useful in the present invention.

In one embodiment, the liposomes comprise about 50-75 mol percent of at least one phosphatidylcholine lipids. In another embodiment, the liposomes comprise about 60-70 mol percent phosphatidylcholine lipid. In another embodiment, the liposomes comprise about 65 mol percent phosphatidylcholine lipid.

In another embodiment, the lipid is a poly(ethylene glycol)-lipid derivative (PEG-lipid). In one embodiment, the PEG-lipid is a diacyl-phosphatidylethanolamine-N-[methoxy(polyethene glycol)]. The molecular weight of the poly(ethylene glycol) in the PEG-lipid is generally in the range of from about 500 Da to about 5000 Da. The poly(ethylene glycol) can have a molecular weight of, for example, 750 Da, 1000 Da, 2000 Da, or 5000 Da. In one embodiment, the PEG-lipid is selected from distearoyl-phosphatidylethanolamine-N-[methoxy(polyethene glycol)-2000] (DSPE-PEG-2000) and distearoyl-phosphatidylethanolamine-N-[methoxy(polyethene glycol)-5000] (DSPE-PEG-5000). In one embodiment, the PEG-lipid is DSPE-PEG-2000.

In one embodiment, the liposomes comprise about 1-10 mol percent of at least one PEG-lipid. In another embodiment, the liposomes comprise about 1-5 mol percent PEG-lipid. In some embodiments, the liposomes comprise about 3 mol percent PEG-lipid.

In one embodiment, the liposomes comprise cholesterol, at least one phosphatidylcholine lipid, and at least one PEG-lipid. In one embodiment, the molar ration of cholesterol to photphatidylcholine lipid to PEG-lipid is about 2:1:0.1. In a particular embodiment, the liposomes comprise cholesterol, photphatidylcholine lipid, and DSPE-PEG-2000. In one embodiment, the molar ratio of cholesterol to photphatidylcholine lipid to DSPE-PEG-2000 is about 2:1:0.1.

The nanolipogels may further comprise a core. In one embodiment, the core includes one or more inhibitors of the invention and at least one host molecule. The inhibitor may be complexed to the host molecules, dispersed within the nanoliposome, or combinations thereof. Host molecules are molecules or materials which reversibly associate with an inhibitor to form a complex. By virtue of their ability to reversibly form complexes with inhibitors, host molecules can function to control the release of a complexed inhibitor in vivo. In one embodiment, the nanolipogel comprises at least one liposome and a core.

In some cases, the host molecule is a molecule that forms an inclusion complex with an inhibitor of the invention. Inclusion complexes are formed when an inhibitor (i.e., the guest) or portion of an active agent inserts into a cavity of another molecule, group of molecules, or material (i.e., the host). Typically, the guest molecule associates with the host molecule without affecting the framework or structure of the host. For example, in the case of inclusion complexes, the size and shape of the available cavity in the host molecule remain substantially unaltered as a consequence of complex formation.

The host molecule may be a small molecule, an oligomer, a polymer, or combinations thereof. Exemplary hosts include polysaccharides such as amyloses, cyclodextrins, and other cyclic or helical compounds containing a plurality of aldose rings, for example, compounds formed through 1,4 and 1,6 bonding of monosaccharides (such as glucose, fructose, and galactose) and disaccharides (such as sucrose, maltose, and lactose). Other exemplary host compounds include cryptands, cryptophanes, cavitands, crown ethers, dendrimers, ion-exchange resins, calixarenes, valinomycins, nigericins, catenanes, polycatenanes, carcerands, cucurbiturils, and spherands.

In still other embodiments, organic host compounds or materials include carbon nanotubes, fullerenes, and/or grapheme-based host materials. Carbon nanotubes (CNTs) are allotropes of carbon with a cylindrical nanostructure. Nanotubes are members of the fullerene structural family, which also includes the spherical buckyballs, and the ends of a nanotube may be capped with a hemisphere of the buckyball structure. Their name is derived from their long, hollow structure with the walls formed by one-atom-thick sheets of carbon, called graphene. These sheets are rolled at specific and discrete (“chiral”) angles, and the combination of the rolling angle and radius decides the nanotube properties. Nanotubes can be categorized as single-walled nanotubes (SWNTs) and multi-walled nanotubes (MWNTs). Nanotubes and/or fullerenes can serve as hosts, for example, by encapsulating or entrapping the material to be delivered (i.e., the guest) within the tubes or fullerenes. Alternatively, the exterior and/or interior of the tubes and/or fullerenes can be functionalized with functional groups which can complex to the guest to be delivered. Complexations include, but are not limited to, ionic interactions, hydrogen bonding, Van der Waals interactions, and pi-pi interactions, such as pi-stacking.

Graphenes are also an allotrope of carbon. The structure of graphene is a one-atom-thick planar sheet of sp²-bonded carbon atoms that are densely packed in a honeycomb crystal lattice. Graphene is the basic structural element of some carbon allotropes including graphite, charcoal, carbon nanotubes and fullerenes. The guest to be delivered can associate with and/or complex to graphene or functionalized graphene as described above for nanotubes and fullerenes.

The host material can also be an inorganic material, including but not limited to, inorganic phosphates and silica.

Suitable host molecules are generally selected for incorporation into nanolipogels in view of the identity of the active agent(s) to be delivered and the desired drug release profile. In order to form a complex with the active agent being delivered, the host molecule is generally selected to be complimentary to the active agent both in terms of sterics (size) and electronics (charge and polarity). For example, in the case of host molecules that form inclusion complexes with the inhibitor to be delivered, the host molecule will typically possess an appropriately-sized cavity to incorporate the active agent. In addition, the host molecule typically possesses a cavity of appropriate hydrophobicity/hydrophilicity to promote complex formation with the inhibitor. The strength of the guest-host interaction will influence the drug release profile of the active agent from the nanolipogel, with stronger guest-host interactions generally producing more prolonged drug release.

Generally, the host molecules are dispersed within the polymeric matrix that forms the nanolipogel core. In some cases, one or more host molecules are covalently coupled to the polymeric matrix. For example, the host molecules may be functionalized with one or more pendant reactive functional groups that react with the polymer matrix. In particular embodiments, the host molecules contain one or more pendant reactive functional groups that react with the polymer matrix to crosslink the polymer matrix. Examples of suitable reactive functional groups include methacrylates, acrylates, vinyl groups, epoxides, thiiranes, azides, and alkynes.

In a particular embodiments, the host molecule is a cyclodextrin. Cyclodextrins are cyclic oligosaccharides containing six (α-cyclodextrin), seven (β-cyclodextrin), eight (γ-cyclodextrin), or more α-(1,4)-linked glucose residues. The hydroxyl groups of the cyclodextrins are oriented to the outside of the ring while the glucosidic oxygen and two rings of the non-exchangeable hydrogen atoms are directed towards the interior of the cavity. As a result, cyclodextrins possess a hydrophobic inner cavity combined with a hydrophilic exterior. Upon combination with a hydrophobic inhibitor, the inhibitor (i.e., the guest) inserts into the hydrophobic interior of the cyclodextrin (i.e., the host).

The cyclodextrin may be chemically modified such that some or all of the primary or secondary hydroxyl groups of the macrocycle, or both, are functionalized with one or more pendant groups. The pendant groups may be reactive functional groups that can react with the polymeric matrix, such as methacrylates, acrylates, vinyl groups, epoxides, thiiranes, azides, alkynes, and combinations thereof. The pendant groups may also serve to modify the solubility of the cyclodextrin. Exemplary groups of this type include sulfinyl, sulfonyl, phosphate, acyl, and C1-C12 alkyl groups optionally substituted with one or more hydroxy, carboxy, carbonyl, acyl, oxy, and oxo groups. Methods of modifying these alcohol residues are known in the art, and many cyclodextrin derivatives are commercially available.

Examples of suitable cyclodextrins include α-cyclodextrin, β-cyclodextrin, γ-cyclodextrin, methyl α-cyclodextrin, methyl β-cyclodextrin, methyl γ-cyclodextrin, ethyl β-cyclodextrin, butyl α-cyclodextrin, butyl β-cyclodextrin, butyl γ-cyclodextrin, pentyl γ-cyclodextrin, hydroxyethyl β-cyclodextrin, hydroxyethyl γ-cyclodextrin, hydroxypropyl-β-cyclodextrin, 2-hydroxypropyl α-cyclodextrin, 2-hydroxypropyl γ-cyclodextrin, 2-hydroxybulyl β-cyclodextrin, acetyl α-cyclodextrin, acetyl β-cyclodextrin, acetyl γ-cyclodextrin, acrylate-β-cyclodextrin, propionyl β-cyclodextrin, butyryl β-cyclodextrin, succinyl α-cyclodextrin, succinyl β-cyclodextrin, succinyl γ-cyclodextrin, benzoyl β-cyclodextrin, palmityl β-cyclodextrin, toluenesulfonyl β-cyclodextrin, acetyl methyl β-cyclodextrin, acetyl butyl β-cyclodextrin, glucosyl α-cyclodextrin, glucosyl β-cyclodextrin, glucosyl γ-cyclodextrin, maltosyl α-cyclodextrin, maltosyl β-cyclodextrin, maltosyl γ-cyclodextrin, α-cyclodextrin carboxymethylether, β-cyclodextrin carboxymethylether, γ-cyclodextrin carboxymethylether, carboxymethylethyl β-cyclodextrin, phosphate ester α-cyclodextrin, phosphate ester β-cyclodextrin, phosphate ester γ-cyclo dextrin, 3-trimethylammonium-2-hydroxypropyl β-cyclodextrin, sulfobutyl ether β-cyclodextrin, carboxymethyl α-cyclodextrin, carboxymethyl β-cyclodextrin, carboxymethyl γ-cyclodextrin, and combinations thereof. In one embodiment, the core comprises at least one cyclodextrin. In one embodiment, the cyclodextrin is selected from the group consisting of acrylate-β-cyclodextrin, hydroxypropyl-β-cyclodextrin, and mixtures thereof. In another embodiment, the core comprises two or more cyclodextrins. In one embodiment, the core comprises a mixture of acrylate-β-cyclodextrin and hydroxypropyl-β-cyclodextrin.

As a further example, the host molecule may also be a material that temporarily associates with an inhibitor via ionic interactions. For example, conventional ion exchange resins known in the art for use in controlled drug release may serve as host molecules. See, for example, Chen, et al. “Evaluation of ion-exchange microspheres as carriers for the anticancer drug doxorubicin: in vitro studies.” J. Pharm. Pharmacol 44(3):211-215 (1992) and Farag, et al. “Rate of release of organic carboxylic acids from ion exchange resins” J. Pharm. Sci. 77(10): 872-875(1988).

In a non-limiting example, when the inhibitor being delivered is a cationic species, suitable ion exchange resins may include a sulfonic acid group (or modified sulfonic acid group) or an optionally modified carboxylic acid group on a physiologically acceptable scaffold. Similarly, where the inhibitor is an anionic species, suitable ion exchange resins may include amine-based groups (e.g., trimethylamine for a strong interaction, or dimethylethanolamine for a weaker interaction). Cationic polymers, such as polyethyleneimine (PEI), can function as host molecules for complex oligonucleotides such as siRNA. In other cases, the host molecule is a dendrimer, such as a poly(amidoamine) (PAMAM) dendrimer. Cationic and anionic dendrimers can function as host materials by ionically associating with inhibitors, as described above. In addition, medium-sized dendrimers, such as three- and four-generation PAMAM dendrimers, may possess internal voids spaces which can accommodate inhibitors, for example, by complexation of nucleic acids.

The core may also comprise at least one photoinitiator. The photoinitiator may catalyzes crosslinking within the nanolipogel between photopolymerizable groups. Non-limiting examples of photoinitiators include Darocur 1173 [2-hydroxy-2-methyl-1-phenyl-1-propanone (HMPP)] and Oligomeric HMPP, Irgacure 184 (1-hydroxy-cyclohexyl-phenylketone), Irgacure 2959 (2-hydroxy-1-[4-(2-hydroxyethoxy)phenyl]-2-methyl-1-propanone), Irgacure 369 (2-benzyl-2-(dimethylamino)-1-[4-(4-morpholinyl)phenyl]-1-butanone), Irgacure 1300 (Irgacure 369+Irgacure 651 (benzildimethylketal)), Irgacure 379 (2-(4-methylbenzyl)-2-(dimethylamino)-1-[4-(4-morpholinyl)phenyl]-1-butanone), Irgacure 127 (2-hydroxy-1-{4-[4-(2-hydroxy-2-methyl-propionyl)-benzyl]-phenyl}-2-methyl-propan-1-one), Irgacure 754 (oxo-phenyl-acetic acid-1-methyl-2-[2-(2-oxo-2-phenyl-acetoxy)-propoxy]-ethyl ester), Irgacure 819 (bis(2,4,6-trimethylbenzoyl)-phenylphosphine oxide), Irgacur 250 (4-isobutylphenyl-4′-methylphenyl iodonium hexafluorophosphate), Darocur ITX (2-isopropylthioxanthone and 4-isopropylthioxanthone), Darocur EDB (ethyl-4-dimethylamino benzoate), Darocur EHA (2-ethylhexyl-4-dimethylamino benzoate), or combinations thereof. In one embodiment, the photoinitiator is an Irgacure photoinitiator.

In another aspect, the nanolipogel contains one or more crosslinkable polymers. Preferably, the crosslinkable polymers contain one or more photo-polymerizable groups, allowing for the crosslinking within the nanolipogel. Examples of suitable photo-polymerizable groups include vinyl groups, acrylate groups, methacrylate groups, and acrylamide groups. Photopolymerizable groups, when present, may be incorporated within the backbone of the crosslinkable polymers, within one or more of the sidechains of the crosslinkable polymers, at one or more of the ends of the crosslinkable polymers, or combinations thereof.

The nanolipogel can be in the form of spheres, discs, rods or other geometries with different aspect ratios. The nanolipogel can be larger, i.e., microparticles. The nanolipogel is typically formed of synthetic or natural polymers capable of encapsulating agents by remote loading and tunable in properties so as to facilitate different rates of release. Release rates are modulated by varying the polymer to lipid ratio from 0.05 to 5.0, more preferably from 0.5 to 1.5.

Nanolipogels may be loaded with an inhibitor either prior to, during or after formation and subsequently function as controlled-release vehicles for the inhibitor. The nanolipogel can be loaded with more than one inhibitor such that controlled release of the multiplicity of inhibitors is subsequently achieved. In one embodiment, the nanolipogel is formed by rehydrating a previously lyophilized liposome in the presence of an inhibitor, a host material, and a photoinitiator.

In one embodiment, the nanolipogel of the invention comprises at least one liposome and a core comprises at least one inhibitor, at least one host material, and at least one photoinitiator. In another embodiment, the nanolipogel of the invention comprises at least one liposome comprising cholesterol, L-α-photphatidylcholine, and DSPE-PEG-2000 and a core comprising BHB, a mixture of acrylate-β-cyclodextrin and hydroxypropyl-β-cyclodextrin, and an Irgacure photoinitiator.

### Methods of the Invention

The invention includes a method of treating or preventing an NLRP3 inflammasome-related disease or disorder in a subject in need thereof. The method comprises administering a therapeutically effective amount of a composition comprising an NLRP3 inflammasome inhibitor to the subject. In one embodiment, the method further comprises administering to the subject an additional therapeutic agent.

Non-limiting examples of NLRP3 inflammasome-related diseases or disorders include gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multisystem inflammatory disease (NOMID), Muckle-Wells Syndrome (MWS) and Familial Cold Autoinflammatory syndrome (FCAS).

In another aspect, the invention includes a method of treating or preventing an NLRP3 inflammasome-related disease or disorder in a subject in need thereof. The method comprises administering to the subject a ketogenic diet. A ketogenic diet (KD) is a high-fat, low-carbohydrate diet that alters the body's metabolism to burn fats in preference to carbohydrates. The use of fats as a primary energy source leads to a state of ketosis and the accumulation of ketone bodies in the blood. Upon transitioning into ketosis, the body begins cleaving fats into fatty acids and glycerol and transforms the fatty acids into acetyl CoA molecules which are then eventually transformed into ketone bodies in the liver. As a result, the increased level of ketone bodies in the blood provides an in vivo method of inhibiting the NLRP3 inflammasome. Ketogenic diets, such as the Atkins diet, have been used clinically to treat children with drug-resistant epilepsy by increasing the levels of ketone bodies, such as BHB, in the blood. Therefore, in one embodiment, the NLPR3 inflammasome may be inhibited by administering to the subject a diet supplemented with a BHB precursor, such as 1,3-butanediol ketone diesters, that gets converted to BHB in the body, thereby increasing the BHB levels in blood. In some embodiments, the ketogenic diet is administered in combination with a composition comprising an NLRP3 inflammasome inhibitor.

In one embodiment, the ketogenic diet is high in dietary fat and low in carbohydrates with moderate levels of protein. In one embodiment, the weight ratio of the fat to the sum of the carbohydrate and the protein is at least 2 to 1. In another embodiment, the weight ratio of the fat to the sum of the carbohydrate and the protein is at least 3 to 1. In another embodiment, the weight ratio of the fat to the sum of the carbohydrate and the protein is at least 4 to 1. In another embodiment, the weight ratio of the fat to the sum of the carbohydrate and the protein is at least 5 to 1.

In another embodiment, the diet is supplemented with ketogenic compounds. Ketogenic compounds are compounds that are converted to ketone bodies, such as BHB, in the body in order to elevate the levels of ketone bodies in the blood. Non-limiting examples of such compounds include medium chain fatty acids such as a medium chain triglycerides (MCT), referring to any glycerol molecule ester-linked to three fatty acid molecules, each fatty acid molecule having a carbon chain of 5-12 carbons, L-carnitine and derivatives thereof, 1,3-butanediol and ketone diesters thereof, ethyl acetoacetate, and ethyl BHB.

In another aspect, the invention includes a method of treating or preventing an NLRP3 inflammasome-related disease or disorder in a subject in need thereof. The method comprises administering a therapeutically effective amount of a composition comprising an NLRP3 inflammasome inhibitor to a joint in the subject. The compound may be administered using any method known in the art. In a non-limiting embodiment, the compound of the invention can be administered in, within, and/or adjacent to a joint or joints of a patient that has, or is at risk of developing an NLRP3 inflammasome-related disease or disorder, such as gouty arthritis, as a treatment strategy to reduce inflammation and neutrophil influx. In one embodiment, the composition is administered by injection directly into, within or adjacent to a joint. In another embodiment, the composition is administered topically on and/or around a joint.

In one embodiment, administering the compound of the invention to the subject allows for administering a lower dose of the therapeutic agent compared to the dose of the therapeutic agent alone that is required to achieve similar results in treating or preventing an NLRP3 inflammasome-related disease or disorder in the subject. For example, in one embodiment, the NLRP3 inflammasome inhibitor enhances the anti-NLRP3 inflammasome activity of the additional therapeutic compound, thereby allowing for a lower dose of the therapeutic compound to provide the same effect.

In one embodiment, the NLRP3 inflammasome inhibitor and the therapeutic agent are co-administered to the subject. In another embodiment, the NLRP3 inflammasome inhibitor and the therapeutic agent are coformulated and co-administered to the subject.

In one embodiment, the methods described herein further comprise inhibiting NLRP3 inflammasome activity.

In one embodiment, the subject is a mammal. In another embodiment, the mammal is a human.

### Pharmaceutical Compositions and Therapies

Administration of an NLRP3 inflammasome inhibitor of the invention in a method of treatment may be achieved in a number of different ways, using methods known in the art. The therapeutic and prophylactic methods of the invention thus encompass the use of pharmaceutical compositions comprising an NLRP3 inflammasome inhibitor to practice the methods of the invention. The pharmaceutical compositions useful for practicing the invention may be administered to deliver a dose of 1 ng/kg/day to 100 mg/kg/day. In one embodiment, the invention envisions administration of a dose that results in a concentration of the compound of the present invention between 1 mM and 10 mM in a mammal, preferably a human.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about 1 μg to about 10 mg per kilogram of body weight of the animal. More preferably, the dosage will vary from about 3 μg to about 1 mg per kilogram of body weight of the animal.

Pharmaceutical compositions that are useful in the methods of the invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically-based formulations.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Controlled- or sustained-release formulations of a composition of the invention may be made using conventional technology, in addition to the disclosure set forth elsewhere herein. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, nanolipogels, or microspheres or a combination thereof to provide the desired release profile in varying proportions. Suitable controlled-release formulations known to those of ordinary skill in the art, including those described herein, can be readily selected for use with the compositions of the invention.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

### Administration/Dosing

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a composition of the invention, alone or in combination with a second pharmaceutical agent; and instructions for using the composition to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject.

### Routes of Administration

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the composition is administered by injection into, within and/or adjacent to a joint. In another embodiment, the composition is administered topically onto and/or near to a joint.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

A pharmaceutical composition of the invention may be prepared, packaged, or sold in a formulation suitable for topical administration. There are several advantages to delivering compounds, including drugs or other therapeutic agents, into the skin (dermal drug delivery) or into the body through the skin (transdermal drug delivery). Transdermal compound delivery offers an attractive alternative to injections and oral medications. Dermal compound delivery offers an efficient way to deliver a compound to the skin of a mammal, and preferably a human, and provides a method of treatment of the skin, or otherwise provides a method of affecting the skin, without the need to break or damage the outer layer of the skin.

A number of compounds, including some drugs, will penetrate the skin effectively simply because the molecules are relatively small and potent at small doses of 0.1 mg to 15 mg/day (Kanikkannan et al., 2000, Curr. Med. Chem. 7:593-608). Many other compounds and drugs can be delivered only when an additional enhancement system is provided to “force” them to pass through the skin. Among several methods of transdermal drug delivery are electroporation, sonophoresis, iontophoresis, permeation enhancers (cyclodextrins), and liposomes. While the aforementioned methods are also included in the present invention for dermal delivery of the compounds of the invention, liposomes represent a preferred dermal delivery method.

The invention encompasses the preparation and use of a dermally-acting composition comprising a compound useful for the treatment or prevention of an autoimmune disorder (e.g. MS). Such a composition may consist of the active ingredient alone, in a form suitable for administration to a subject, or the composition may comprise at least one active ingredient and one or more pharmaceutically acceptable carriers, one or more additional ingredients, or some combination of these. The active ingredient may be present in the composition in the form of a physiologically acceptable ester or salt, such as in combination with a physiologically acceptable cation or anion, as is well known in the art.

In one aspect, a dermal delivery vehicle of the invention is a composition comprising at least one first compound that can facilitate dermal delivery of at least one second compound associated with, or in close physical proximity to, the composition comprising the first compound. As will be understood by the skilled artisan, when armed with the disclosure set forth herein, such delivery vehicles include, but should not be limited to, liposomes, nanosomes, phosopholipid-based non-liposome compositions (eg., selected cochleates), among others.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.001% to about 90% (w/w) active ingredient, although the concentration of the active ingredient may be as high as the solubility limit of the active ingredient in the solvent. Formulations for topical administration may further comprise one or more of the additional ingredients described herein.

In one aspect of the invention, a dermal delivery system includes a liposome delivery system, and that the present invention should not be construed to be limited to any particular liposome delivery system. Based on the disclosure set forth herein, the skilled artisan will understand how to identify a liposome delivery system as being useful in the present invention.

The present invention also encompasses the improvement of dermal and transdermal drug delivery through the use of penetration enhancers (also called sorption promoters or accelerants), which penetrate into skin to reversibly decrease the barrier resistance. Many compounds are known in the art for penetration enhancing activity, including sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes. Other enhancers include oleic acid, oleyl alcohol, ethoxydiglycol, laurocapram, alkanecarboxylic acids, dimethylsulfoxide, polar lipids, or N-methyl-2-pyrrolidone.

In alternative embodiments, the topically active pharmaceutical or cosmetic composition may be optionally combined with other ingredients such as moisturizers, cosmetic adjuvants, anti-oxidants, chelating agents, surfactants, foaming agents, conditioners, humectants, wetting agents, emulsifying agents, fragrances, viscosifiers, buffering agents, preservatives, sunscreens and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art.

In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art. The compositions of this invention may also contain active amounts of retinoids (i.e., compounds that bind to any members of the family of retinoid receptors), including, for example, tretinoin, retinol, esters of tretinoin and/or retinol and the like.

The composition of the invention may comprise a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition of the invention such as a therapeutic gel or cream. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound for use in the invention in the aqueous gel formulation. Preferred antioxidants for some compounds are BHT, BHA, alpha-tocopherol and ascorbic acid in the preferred range of about 0.01% to 5% and BHT in the range of 0.01% to 1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Additional components may include, but should not be limited to those including water, oil (e.g., olive oil/PEG7), biovera oil, wax (e.g., jojoba wax), squalene, myristate (e.g., isopropyl myristate), triglycerides (e.g., caprylic triglyceride), Solulan 98, cocoa butter, shea butter, alcohol (e.g., behenyl alcohol), stearate (e.g., glycerolmonostearate), chelating agents (e.g., EDTA), propylene glycol, SEPIGEL (Seppic, Inc., Fairfield, N.J.), silicone and silicone derivatives (e.g., dimethicone, cyclomethicone), vitamins (e.g., vitamin E), among others.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease. Using a wax/pH-sensitive polymer mix, a gastric insoluble composition may be obtained in which the active ingredient is entrapped, ensuring its delayed release.

### Buccal Administration

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Vaginal Administration

A pharmaceutical composition of the invention may be prepared, packaged, or sold in a formulation suitable for vaginal administration. With respect to the vaginal or perivaginal administration of the compounds of the invention, dosage forms may include vaginal suppositories, creams, ointments, liquid formulations, pessaries, tampons, gels, pastes, foams or sprays. The suppository, solution, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention may be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit may be fabricated to disintegrate rapidly or over a period of several hours. The time period for complete disintegration may be in the range of from about 10 minutes to about 6 hours, e.g., less than about 3 hours.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives.

### Additional Administration Forms

Additional dosage forms of this invention include dosage forms as described in U.S. Pat. Nos. 6,340,475; 6,488,962; 6,451,808; 5,972,389; 5,582,837 and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Kits of the Invention

The invention also includes a kit comprising a an NLRP3 inflammasome inhibitor and an instructional material that describes, for instance, administering the NLRP3 inflammasome inhibitor to a subject as a prophylactic or therapeutic treatment or a non-treatment use as described elsewhere herein. In an embodiment, the kit further comprises a (preferably sterile) pharmaceutically acceptable carrier suitable for dissolving or suspending the therapeutic composition, comprising an NLRP3 inflammasome inhibitor, for instance, prior to administering the molecule to a subject. Optionally, the kit comprises an applicator for administering the inhibitor.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Ketone Body β-Hydroxybutyrate Blocks NLRP3 Inflammasome

The results described herein demonstrate that β-hydroxybutyrate (BHB), but neither acetoacetate nor butyrate, suppresses the inflammasome in response to diverse NLRP3 proinflammatory inducers without deactivating the NLRC4, AIM2 or non-canonical caspase-11 inflammasomes. It was found that BHB's inhibitory effects on the NLRP3 inflammasome activation were not dependent on classical starvation regulated mechanisms like AMPK, ROS, autophagy or glycolytic inhibition. Furthermore, BHB blocked NLRP3 independently of mitochondrial uncoupling or oxidation for energetic purposes, without requirement for GPR109a or histone acetylation. The chiral enantiomer (S)-BHB, which is not normally produced during ketogenesis and does not get oxidized via TCA cycle, also blocks NLRP3. Without being bound by any particular theory, this result suggests that (S)-BHB may have an improved therapeutic window due to its longer half-life. It was observed that BHB's major mechanism of anti-inflammasome action involves preventing the K⁺ efflux and ASC oligomerization in response to NLRP3 activators. Without being bound by any particular theory, this result suggests that during energy deficit BHB can dampen NLRP3 sensing without ATP utilization in macrophages thus allowing energy allocation for essential functioning of heart and brain. BHB was found to deactivate the inflammasome in human monocytes and in mouse models of urate induced inflammation and NLRP3 driven autoinflammatory diseases like Muckle-Wells Syndrome (MWS) and Familial Cold Autoinflammatory syndrome (FCAS).

The materials and methods employed in these experiments are now described.

### Materials and Methods

### Mice and Animal Care

The global Nlrp3^{-/-}, Gpr109a^{-/-}, Ucp2^{-/-} and Sirt2^{-/-} knockout mice have been previously described (Vandanmagsar et al., 2011, Nat. Med. 17:179-188; Youm et al., 2013, Cell Metab. 18:519-532; Kayagaki et al., 2013, Science 341:1246-1249). The, Oxct1 floxed mice (Cotter et al., 2013, J. Biol. Chem. 288:19739-19749) and Atg5 floxed mice were crossed with LysM-Cre animals for macrophage specific gene ablations. NLRP3^{L351P} gain of function Familial Cold Autoinflammatory Syndrome (FCAS) and NLRP3^{A350V} Muckle-Wells Syndrome (MWS) knockin mutation have been previously described (Yu et al., 2006, Cell. Death Differ. 13:263-249; Demento et al., 2011, Trends Biotechnol. 29:294-306). Briefly, the Nlrp3^{L351PneoR/+}and Nlrp3^{A350VPneoR/+}mutation was conditionally activated by breeding these animals with tamoxifen-inducible Cre mice (B6.Cg-Tg(CAG-cre/Esr1*)5Amc/J) or in vitro by treating cells with 4-hydroxy tamoxifen. Mice were fed 1,3-butanediol ketone diesters (KD) for one week after weaning and injected with tamoxifen for 3 days and analysed. The WT littermates and mutant cohorts were housed with a 12-hour light/12-hour dark cycle at 22° C. The mice were multi-housed and were either fed ad libitum normal chow diet consisting of 4.5% fat (5002; LabDiet) or ad libitum normal chow diet mixed in with 20% 1,3-butanediol ketone diesters and aged in the specific-pathogen free barrier facility in ventilated cage racks that delivers HEPA filtered air to each cage with free access to sterile water through a hydropac system. Sentinel mice in the animal rooms were negative for currently tested standard murine pathogens (Ectromelia, EDIM, LCMV, Mycoplasma pulmonis, MHV, MNV, MPV, MVM, PVM, REO3, TMEV and Sendai virus) at various times while the studies were performed (RADIL, Research Animal Diagnostic Laboratory, Columbia, Mo.). All experiments and animal use were conducted in compliance with the National Institutes of Health Guide for the Care and Use of Laboratory Animals and were approved by the Institutional Animal Care and Use Committee at Yale and Washington University.

### Human Monocytes

The cryopreserved peripheral blood monocuclear cells were used to sort CD14+ monocytes using isolation kit from Miltenyi (130-091-153). A total of 6 healthy subjects, (67 y female, 31 y female, 44 y female, 67 y male, 31 y male and 35 y male) were used for monocyte isolations. Monocytes were seeded in 6 well plates at concentration of 3 million/mL of RPMI1640 media with 10% FCS and antibiotic/antimycotic mixture and stimulated with LPS for 4 h. The supernatants were used to measure IL-1β, IL-18 and TNF using ELISA. Human peripheral blood was collected by the Health Apheresis Unit and the Clinical Core Laboratory, the National Institute on Aging-Intramural Research Program, under Human Subject Protocol #2003054 and Tissue Procurement Protocol #2003-071.

### Cell Culture

All steps were performed using sterile technique. Femurs were collected in RPMI (Life Technologies, Grand Island, N.Y.)+5% FBS (R5; Omega Scientific, Tarzana, Calif.). Both ends of the femur were then cut and the femur was flushed with R5. The bone marrow was centrifuge at 450 g for 5 min, the supernatant was decanted and red blood cells were lysed using ACK lysis buffer (Quality Biological, Gaithersburg, Md.). After neutralization with R5, bone marrow cells were centrifuged, resuspended in 10 ml of R5 and placed into a 6 well plate. Non-adherent cells were collected the following morning. The non-adherent cells were resuspended at 4×10⁶ cells/ml in media consisting of 10 ml supernatant of non-adherent cells, 7.2 ml L929 conditioned media, 6.8 ml R5 and MCSF (long/ml; R&D Systems, Minneapolis, Minn.). An additional 2 ml of fresh media was added 4 d after isolation. Non-adherent cells were collected on day 7, separated by density gradient separation using Fico/Lite (Atlanta Biologicals, Flowery Branch, Ga.) and mononuclear cells were collected. Cells were rinsed twice with Dulbecco's PBS+2% FBS, and resuspended at 1×10⁶ cells/ml. Cells were treated with ultrapure LPS (Invivogen, San Diego, Calif.) alone or in combination with 5 mM ATP (Sigma, St. Louis, Mo.) or 200 μM palmitate-BSA (Sigma). The BMDMs were also primed with ultrapure lipid A (10 μg/ml; Invivogen, San Diego, Calif.), lipoteichoic acid (10 μg/ml; Invivogen), or Pam3-CSK4 (10 μg/ml; Invivogen) for 4 hour and stimulated with various NLRP3 activators (ATP 5 mM; Sigma, MSU 250 ug/ml; Invivogen, Silica; 200 ug/ml; Invivogen, sodium palmitate; 200 μM; Sigma, ceramide C6 80 μg/ml; Cayman, and sphingosine 40 μM; Cayman, Ann Arbor, Mich.) for 1 hour together with D-BHB or L-(BHB) at indicated concentration. The cell supernatants and cell lysates were collected 1 h after BHB treatment and analyzed for caspase-1 and IL-1β.

Salmonella typhimurium (SL1344) and Francisella tularensis was grown overnight and then subcultured to mid-Log phase. BMDMs were infected with 1 MOI S. typhimurium and treated 1 h after infection with 0, 1, 5, or 10 mM of BHB. Cell supernatants and cell lysates were collected 3 h after treatment (4 h after infection).

### ASC Oligomerization and ASC Speck Formation

The ASC oligomerization was performed using previously described methods (Yu et al., 2006, Cell. Death Differ. 13:236-249). BMDM were plated on chamber slides and allowed to attach overnight. The following day cells were primed with LPS and treated with ATP±BHB (10 mM) as described in the materials and methods. Cells were fixed with 4% paraformaldehyde followed by ASC (Enzo Lifesciences) and DAPI staining. ASC specks were quantified using ImageJ software. At least 5 distinct fields were analyzed and a minimum of 550 cells from each treatment condition were quantified. Data are shown as mean±SD and are representative of two independent experiments. Statistical differences were calculated by student's t-test.

### Neutrophil Chemotaxis Assay

Neutrophils were isolated from mouse bone marrow (Stemcell Technologies) and 2×10⁵ cells were plated in 3 μm transwell 96-well plates. Untreated (UnRx) cells had just RPMI (+10% FCS) in the bottom chamber. To induce chemotaxis, LPS+ATP-stimulated macrophage-conditioned media (CM) was diluted 1:1 with RPMI±BHB (10 mM) as indicated. Cells were incubated at 37° C. for 90 minutes and cells that passed through the membrane to the lower chamber were counted. Each point represents an individual mouse. Data are shown as mean±SEM and are pooled from two independent experiments. Statistical differences were calculated by paired 1-way ANOVA.

### Western Blot Analysis

The BMDM cell lysates were prepared using RIPA buffer and immediately snap frozen in liquid nitrogen. Samples were left on ice for 1 hour with vortexing every 10 min. Samples were then centrifuge at 14,000 g for 15 min, the supernatant was collected and the protein concentration was determined using the DC Protein Assay (Bio-RAD). The immunoblot analysis was performed using previously described methods (Vandanmagsar et al., 2011, Nat. Med. 17:179-188). The immune complexes were visualized by incubation with horseradish peroxidase-conjugated anti-rat or anti-rabbit secondary antibody (Amersham Biosciences). Immunoreactive bands were visualized by enhanced chemiluminescence (PerkinElmer Life Sciences).

### Gene Expression Analysis

Total RNA was extracted using the trizol method and transferred to the Qiagen RNeasy mini kit and purified according to the manufacturer's instruction. On the columns, DNA digestion was performed to remove DNA. Synthesis of cDNA and Q-PCR was performed using previously described methods (Martinon et al., 2006, Nature 440:237-241). The primer pairs used for real-time PCR:

Bdh1 (Forward: GCTTCCTTGTATTTGCTGGC (SEQ ID NO: 1),
Reverse: TTCTCCACCTCTTCACTGTTG (SEQ ID NO: 2),
Probe: TGGATGGTTCTCAGTCGGTCACTCT (SEQ ID NO: 3)),
Bdh2 (Forward: TCTCAATGAATCTCAACGTCCG (SEQ ID NO:
4), Reverse: ATCTGTTCTCCACCCCTTTG (SEQ ID NO: 5),
Probe: ATCAACATGTCGTCTGTGGCCTCC (SEQ ID NO: 6)),
Acat1 (forward: GGCTGCTGCAGGAAGTAAGA (SEQ ID NO:
7), Reverse: ATCCCTGCCTTCTCAATGGC (SEQ ID NO: 8)),
Hmgcl (Forward: CAGGTGAAGATCGTGGAAGTC (SEQ ID
NO: 9), Reverse: TGGGAGAAACAAAGCTGGTG (SEQ ID NO:
10) and Gapdh (Forward: TCA ACA GCA ACT CCC ACT
CTT CCA (SEQ ID NO: 11), Reverse: ACC CTG TTG CTG
TAG CCG TAT TCA (SEQ ID NO: 12)).

BHB-Nanolipogel (nLG) Generation and Treatment

nLG is a nanoparticle that combines the advantages of both liposomes and polymer-based particles and it can provide means for delivery of two or more pharmaceutical agents at different rates, especially agents with different chemical properties and or molecular weights. nLGs were fabricated by remotely loading liposomes with BHB and cross-linkable poly(ethylene glycol) oligomers. To prepare liposomes, a molar ratio mixture of 2:1:0.1 phosphatidylcholine/cholesterol/DSPE-PEG(2000)-COOH in chloroform was evaporated under a nitrogen gas stream and then lyophilized after extrusion. Lyophilized liposomes were rehydrated with the aqueous BHB-cyclodextrin-Irgacure-PEG mixture. The phosphatidylcholine used was L-α-phosphatidylcholine. Two cyclodextrins were used in the study. One was acrylate-β-cyclodextrin that is formed from the conjugation of carboxymethyl-β-cyclodextrin sodium salt with 2-aminoethyl methacrylate. The other cyclodextrin was hydroxypropyl-β-cyclodextrin. Vigorous mixing was applied for 30 minutes. The liposomes were then cross-linked under a 430 W UV lamp with UVA light (315-400 nm transmission filter) for 8 minutes on ice to form the nLGs, rinsed with PBS, and pelleted by ultracentrifugation. nLGs were stored at −20° C. until use.

The mice were given BHB-nLG injections (at 5 and 10 mM dose) one day prior to MSU i.p injection at the dose of 2-4 mg/kg bw. The mice were sacrificed after 4 h post MSU injection and peritoneal lavage was performed to collect the infiltrating leukocyte for FACS analysis.

### BMDM Cell Proliferation and Intracellular K⁺ Measurement

The BMDM proliferation in response to BHB treatment was analyzed using the MTT assay according to manufacturer's instructions. The BMDM were incubated with Asante Potassium Green 1 (APG-1) which is a fluorescent indicator with a Kd for measuring cytosolic K+ concentration. It has non-ratiometric large fluorescence dynamic range allows sensing of even small changes in K+ concentration. Optimal excitation was recorded at 517 nm. In addition, the K+ was measured in BMDMs using an ElementXR or Agilent 7700 Inductively Coupled Mass Spectrometry (ICP-MS) using previously described methods (Muñoz-Planillo et al., 2013, Immunity 38:1142-1153). For error analysis, samples and standard solutions were run in duplicate.

### Enzyme-Linked Immunosorbent Assay

The peritoneal cells were incubated overnight in RPMI1640 containing 10% FCS and 1% antibiotic mixture. The supernatants were analyzed for IL-1β. The sera from mice were collected and stored at −80° C. and used for IL-1β (eBiosciences). ELISA was performed according to manufacturer's instructions.

### Flow Cytometry

The peritoneal cells were collected using cold sterile PBS and were stained for CD45, Ly6C, Ly6G and Gr1 and analyzed using FACS Calibur. All the FACS data were analyzed by post collection compensation using FlowJO (Treestar Inc) software.

### Statistical Analyses

A two-tailed Student's t test was used to test for differences between genotypes or treatments; *p<0.05. The results are expressed as the mean±SEM. The differences between means and the effects of treatments were determined by one-way ANOVA using Tukey's test (Sigma Stat), which protects the significance (p<0.05) of all pair combinations.

The results of the experiments are now described.

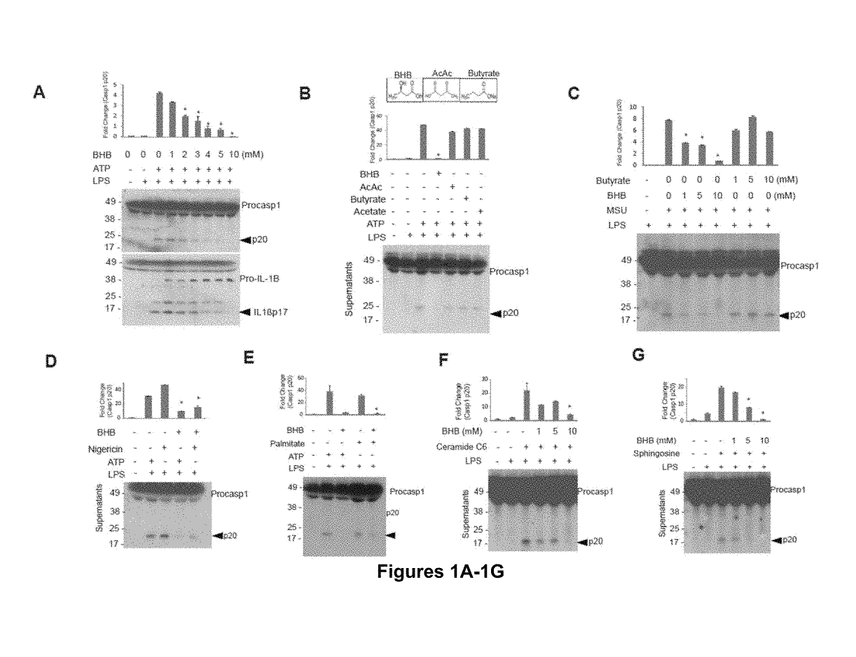

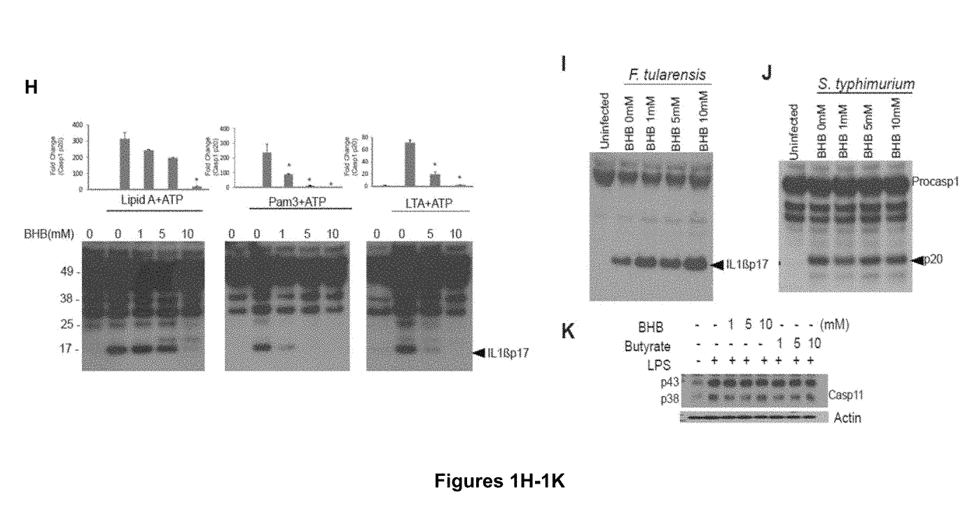

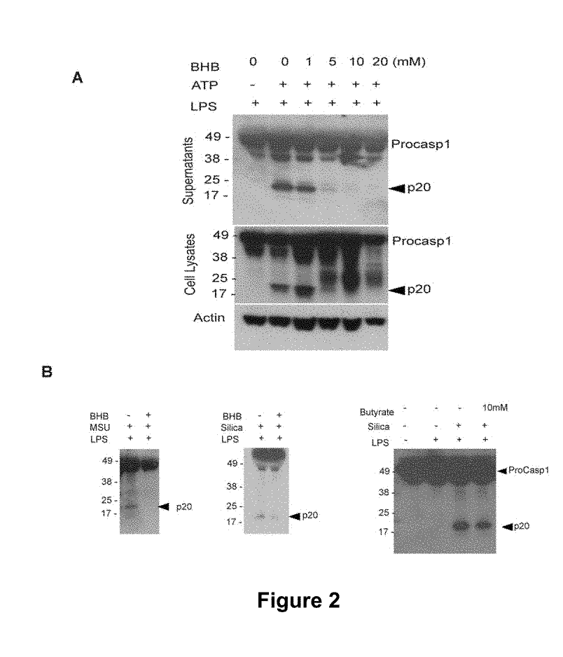

Circulating levels of BHB can increase up to 6-8 mM upon prolonged fasting as liver glycogen stores get utilized (Newman and Verdin, 2014, Trends Endocrinol. Metab. 25:42-52; Cotter et al., 2013, Am. J. Physiol. Heart Circ. Physiol. 304:H1060-1076). To test whether BHB impacts inflammasome activation, LPS primed bone marrow derived macrophages (BMDMs) were treated with NLRP3 activator ATP and BHB for 60 minutes, and caspase-1 activation was then measured by Western blot that detects enzymatically active p20 subunits. BHB dose-dependently inhibited ATP-induced caspase-1 cleavage and processing of biologically active p17 form of IL-1β at concentrations similar to that achieved by strenuous exercise or 2 days fasting (Newman and Verdin, 2014, Trends Endocrinol. Metab. 25:42-52; Cotter et al., 2013, Am. J. Physiol. Heart Circ. Physiol. 304:H1060-1076) (FIGS. 1A and 2A). Ketone body acetoacetate (AcAc) and microbiota-derived short chain fatty acids (SCFAs), butyrate and acetate that are structurally related to BHB, did not affect ATP-induced NLRP3 activation (FIG. 1B). It was then examined whether BHB specifically targets ATP-induced inflammasome activation or common signalling mechanisms in response to structurally diverse NLRP3 activators. BHB, but not butyrate-inhibited monosodium urate (MSU) crystals or particulate matter, induced caspase-1 activation (FIGS. 1C and 2B). Furthermore, BHB blocked inflammasome activation by five additional NLRP3 activators nigericin (FIG. 1D), silica particles (FIG. 2B), lipotoxic fatty acids palmitate (FIG. 1E), ceramides (FIG. 1F), and sphingosine (FIG. 1G). Substituting LPS with different TLR4 pathogen associated molecular pattern (PAMP) agonist lipid A, TLR1/2 ligand Pam3-CSK4, or TLR2 agonist lipoteichoic acid (LTA) induced inflammasome activation that was also effectively suppressed by BHB (FIG. 1H).

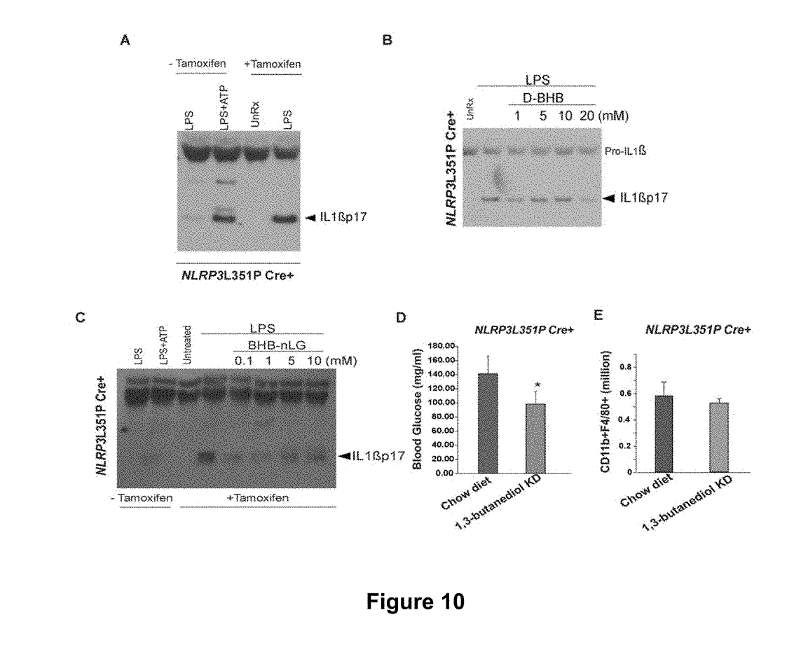

The specificity of inhibitory effects of BHB on other inflammasomes was investigated. The BMDMs were infected with Francisella tularensis to activate the AIM2 and Salmonella typhimurium to activate the NLCR4 inflammasome. BHB did not inhibit either AIM2 inflammasome induced IL-1β activation (FIG. 10 or NLRC4 mediated caspase-1 cleavage (FIG. 1J). Because inflammasomes can also be activated by LPS through caspase-11 activation independently of TLR4 (Kayagaki et al., 2013, Science 341:1246-1249; Hagar et al., 2013, Science 341:1250-1253), the specificity of BHB on the non-canonical inflammasome pathway was examined. The results demonstrate that neither butyrate nor BHB blocks the caspase-11 activation (FIG. 1K). Although not wishing to be bound by any particular theory, these results suggest that BHB controls a central common signalling event that specifically deactivates the NLRP3 inflammasome in response to PAMPs and a wide array of pro-inflammatory DAMPs.

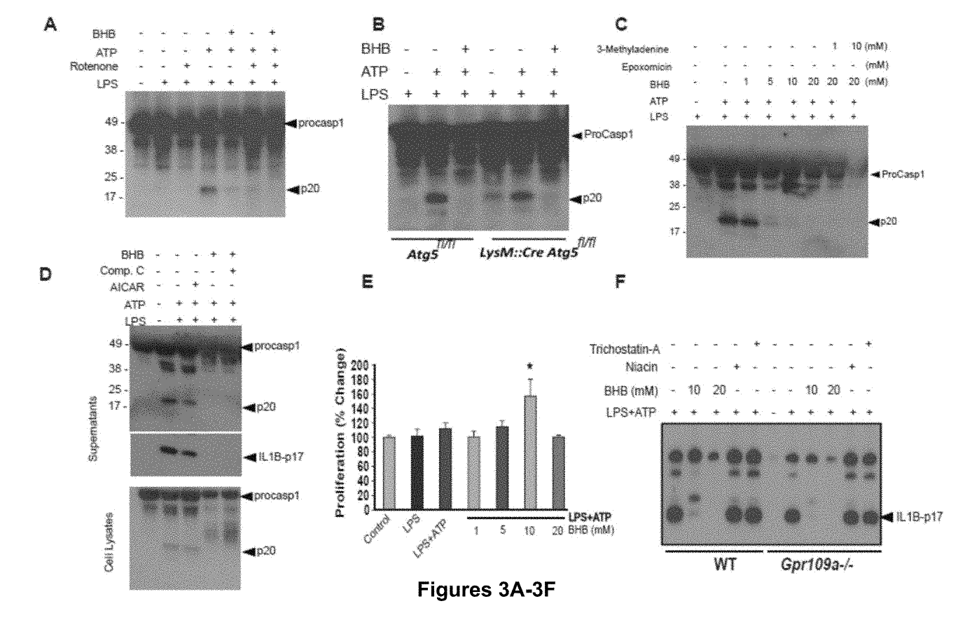

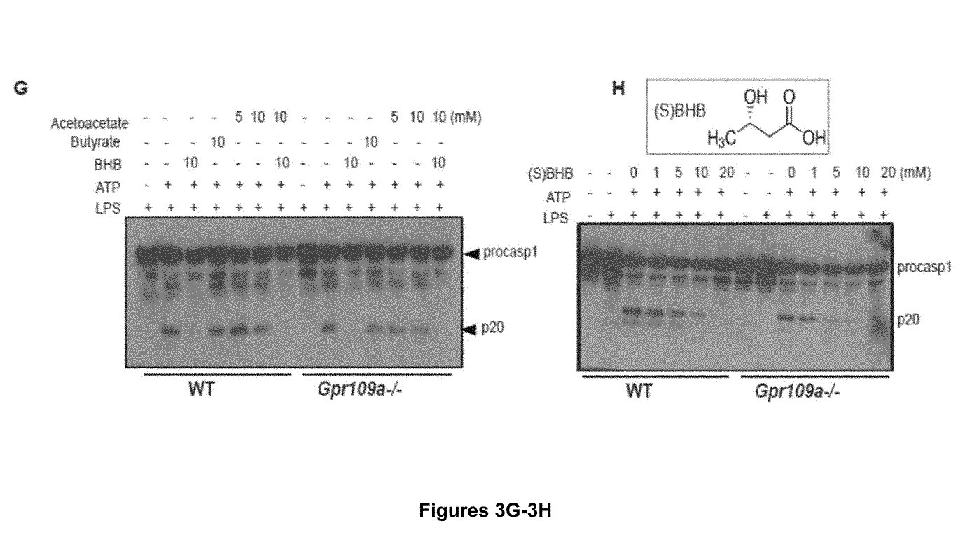

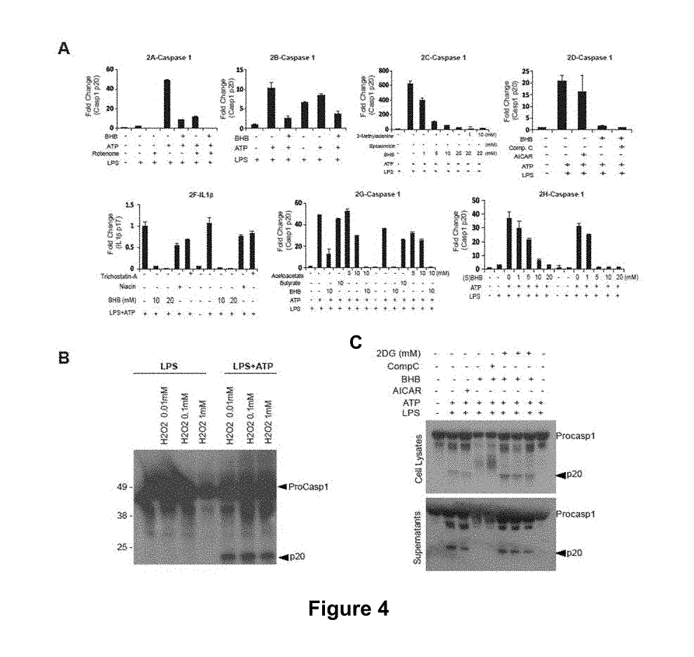

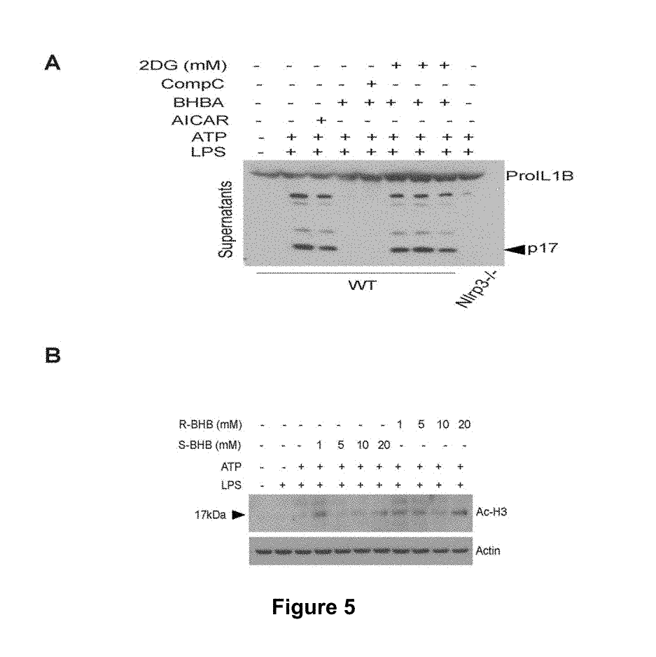

Prolonged fasting and subsequent increase in circulating BHB is linked to reduction in oxidative stress (Shimazu et al., 2013, Science 339:211-214) and an increase in AMPK activity (Laeger et al., 2012, J. Endocrinol. 213:193-203) and autophagy (Finn and Dice, 2005, J. Biol. Chem. 280:25864-25870). Furthermore, all these mechanisms have also been implicated in regulating the NLRP3 inflammasome (Lamkanfi and Dixit, 2014, Cell 157:1013-1022). In an effort to understand the mechanism of BHB's anti-inflammasome effects, the contribution of these pathways towards BHB's anti-inflammasome action was determined. Consistent with recent data (Muñoz-Planillo et al., 2013, Immunity 38:1142-1153), ROS damage via rotenone or hydrogen peroxide (FIGS. 3A, 4A, and 4B) was not sufficient to induce the caspase-1 cleavage and did not prevent BHB's suppressive effects on ATP-induced NLRP3 inflammasome activation. The macrophages deficient in autophagy regulator Atg5 displayed caspase-1 activation by LPS priming alone (FIGS. 3B and 4A). However, the absence of Atg5 mediated autophagy in macrophages was not required for BHB's inhibitory effects on the inflammasome (FIGS. 3B and 4A). Consistent with these findings, autophagy inhibitor 3-methyladenine (3MA) and proteasome blocker epoxomicin did not abrogate BHB's suppressive effects on ATP-induced NLRP3 inflammasome activation (FIGS. 3C and 4A). In addition, BHB suppressed ATP-induced caspase-1 (FIG. 3D) and IL-1β (FIG. 5A) activation in macrophages without involvement of AMPK, and inhibition of glycolysis by 2DG did not mimic BHB's anti-inflammasome effect (FIGS. 3D, 4A, 4C, and 5A). Importantly, BHB did not impair the viability of BMDMs and significantly increased the cellular proliferation at a 10 mM concentration (FIG. 3E). Although not wishing to be bound by any particular theory, these data suggest that BHB functions as a unique signaling entity to deactivate the inflammasome.

It has been suggested that BHB can serve as signalling molecule via the ligation of G protein coupled receptor GPR109a (Taggart et al., 2005, J. Biol. Chem. 280:26649-26652) or by serving as a histone deacetylase (HDAC) inhibitor (Shimazu et al., 2013, Science 339:211-214). When LPS primed and NLRP3 agonist treated macrophages were incubated with HDAC inhibitor trichostatin A (TSA), no effects on inflammasome activation were observed (FIGS. 3F and 4A) despite BHB inducing the H3 acetylation in macrophages (FIG. 5B). To understand the role of GPR109a in BHB's effects on macrophages, niacin, a GPR109a ligand that has been reported to inhibit colonic inflammation (Singh et al., 2014, Immunity 40:128-139), was used. It was observed that unlike BHB, niacin did not block the NLRP3 inflammasome activation (FIGS. 3F and 4A). Finally, BHB's anti-inflammasome effects could not be abrogated in BMDMs deficient in GPR109a (FIGS. 3F, 3G, and 4A). In addition, it was confirmed confirmed that neither butyrate nor acetoacetate affect the NLRP3 inflammasome and GPR109a was not involved (FIGS. 3G and 4A). BHB is a chiral compound and its enantiomeric form (S)-BHB does not enter the TCA cycle but binds Gpr109a with high affinity (Taggart et al., 2005, J. Biol. Chem. 280:26649-26652). It was observed that the (S)-BHB enantiomer retains anti-inflammasome activity similar to bioactive D-(BHB) and does not require Gpr109a to block NLRP3 (FIG. 3H).

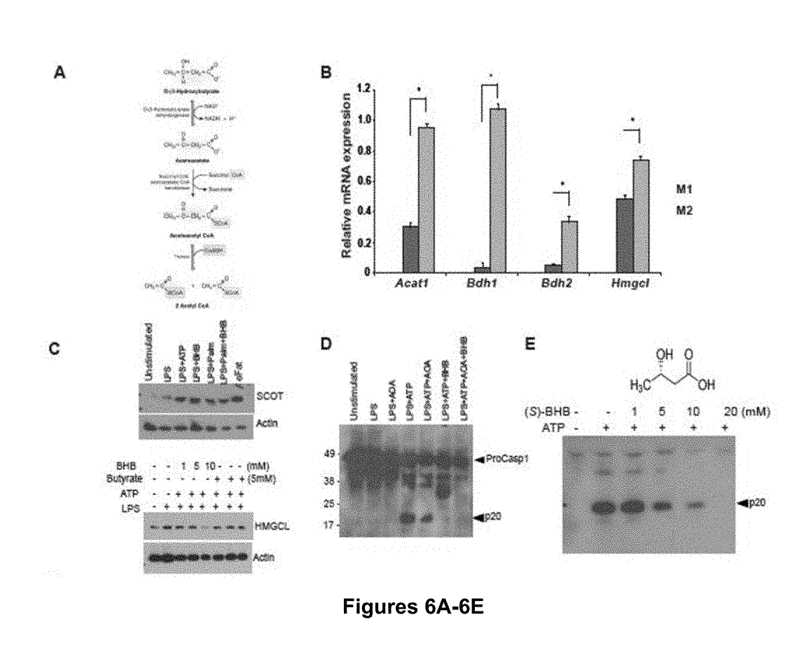

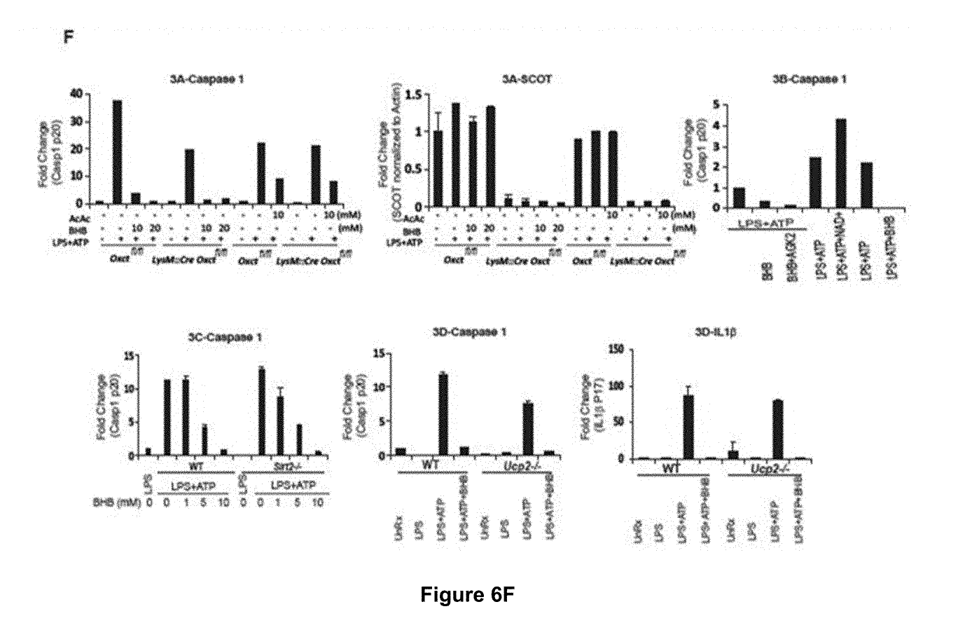

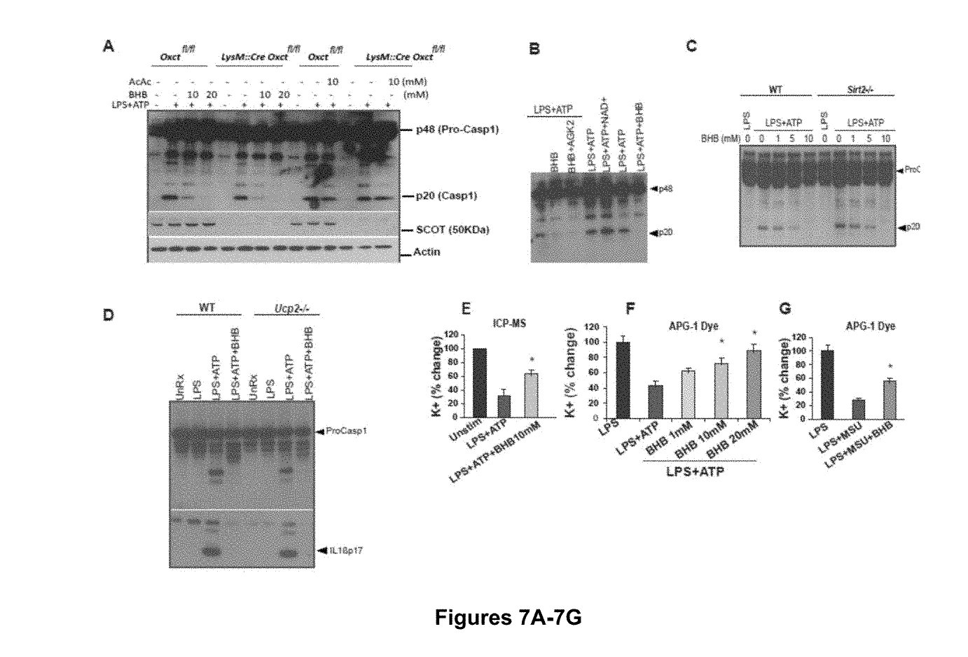

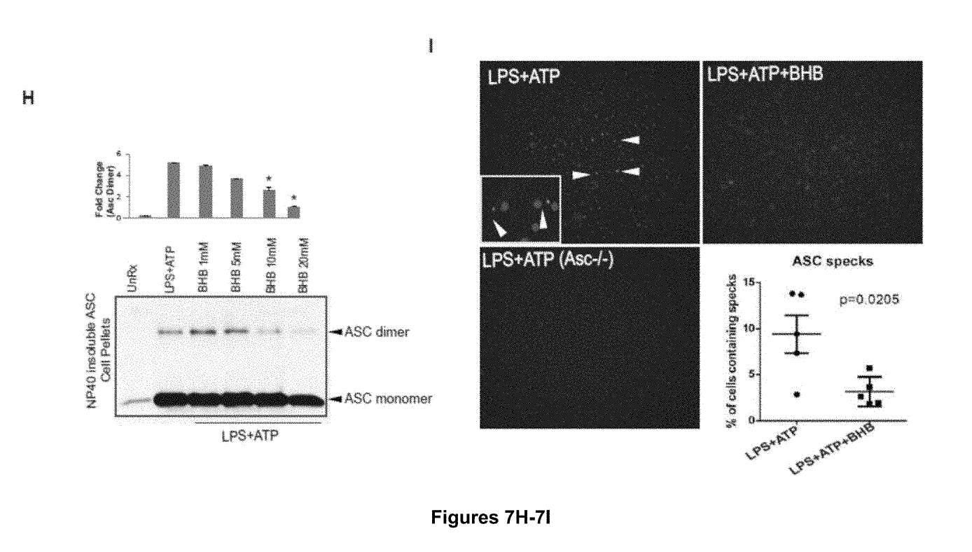

Compared to fatty acids, oxidation of BHB is energetically more efficient as all reducing equivalents generated by ketone oxidation are delivered through NADH to complex-I within the mitochondrial electron transport chain (Cotter et al., 2013, Am J Physiol Heart Circ Physiol. 304:H1060-1076). Furthermore, ketone oxidation increases the redox span between complex-I and complex-III by keeping mitochondrial ubiquinone oxidized Cotter et al., 2013, J. Biol. Chem. 288:19739-19749). It was then examined whether BHB oxidation, entry into TCA, or reduced mitochondrial stress controls its anti-inflammasome action. Consistent with the idea that BHB affects the innate immune compartment, it was observed that macrophages expressed the ketogenic and ketolytic enzymes (FIGS. 6A, 6B, and 6C). Furthermore, compared to M2, the classically activated M1 macrophages showed reduction in Acat1, Bdh1, Bdh2 and Hmgcl expression. Although not wishing to be bound by any particular theory, these results suggest that ketones may affect macrophage polarization (FIG. 6B). In addition, LPS induced the protein expression of key ketolytic enzyme succinyl-CoA:3-oxoacid CoA transferase (SCOT; encoded by Oxct) and ketogenic enzyme HMGCL in BMDMs (FIG. 6C). However, TCA entry inhibitor aminoxyacetate (AOA) did not affect BHB's anti-inflammasome action (FIG. 6D). Furthermore, enantiomer (S)-BHB, which does not enter TCA, efficiently blocked the NLRP3 inflammasome activation (FIG. 6E). To gain definitive evidence whether BHB oxidation controls inflammasome activation, the ketolytic mitochondrial enzyme SCOT (encoded by Oxct1; Singh, 2014, Immunity 40:128-139) was specifically deleted in macrophages (FIGS. 7A and 6F). It was found that TCA intermediates generated through ketone body oxidation in macrophage mitochondria do not mediate the suppressive effects on the NLRP3 inflammasome (FIGS. 7A and 6F).

NAD dependent deacetylase Sirt2 has been implicated in regulating acetylation of α-tubulin that controls microtubule driven apposition of Nlrp3 and Asc (Misawa et al., 2013, Nat. Immunol. 14:454-460). Accordingly, the inhibition of Sirt2 by small molecule AGK2 results in activation of Nlrp3 inflammasome and supplementation with NAD⁺ lowered IL-1β secretion from macrophages (Misawa et al., 2013, Nat. Immunol. 14:454-460). Similar to the data demonstrating that BHB does not require the mitochondrial TCA cycle to elicit its effects on the inflammasome, it was observed that AGK2 did not abrogate BHB's inhibitory effects on the inflammasome, and addition of NAD⁺ did not block caspase-1 activation in response to LPS and ATP (FIGS. 6F and 7B). Furthermore, ablation of Sirt2 or uncoupling protein 2 (UCP2) in macrophages did not control BHB's inhibitory effects on NLRP3 activation (FIGS. 6F, 7C, and 7D), demonstrating that mitochondrial ROS does not play a major role in ketone body's anti-inflammasome effects.

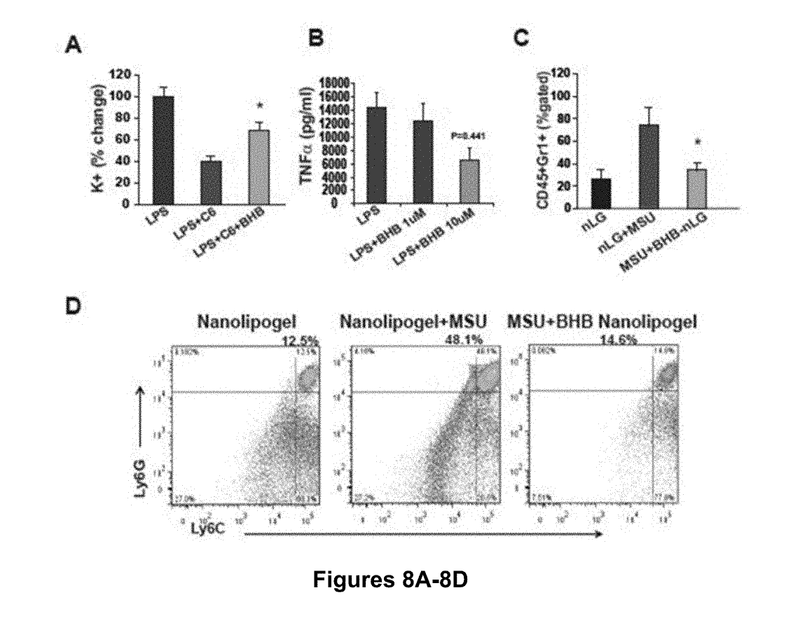

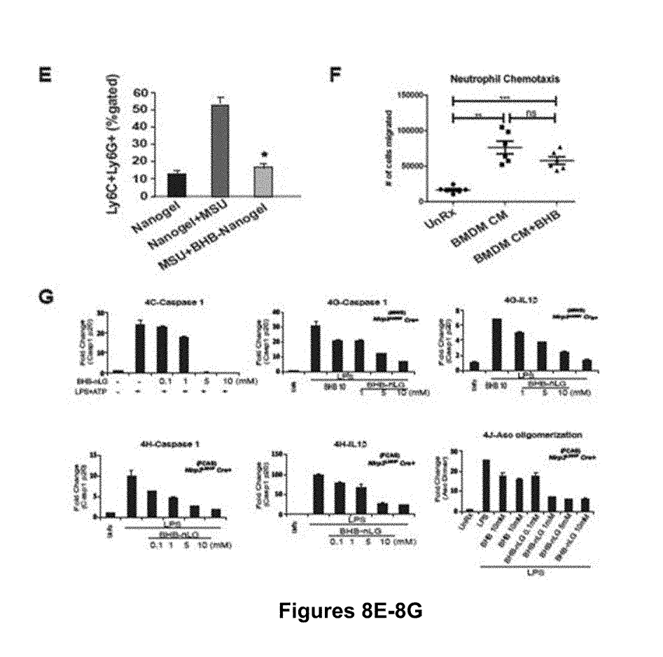

It has been observed that BHB is a strongly anionic endogenous molecule (Cotter et al., 2013, Am J Physiol Heart Circ Physiol. 304:H1060-1076) and exerts anti-epileptic effects by reducing neuronal excitability through regulating intracellular potassium cations (Lutas and Yellen, 2013, Trends Neurosci. 36:32-40). Consistent with recent studies that show K⁺ efflux as a common triggering event for NLRP3 inflammasome activation (Lamkanfi and Dixit, 2014, Cell 157:1013-1022; Muñoz-Planillo et al., 2013, Immunity 38:1142-1153), it was observed that BHB prevented the decline in intracellular K⁺ in response to NLRP3 activators, ATP, MSU and ceramides (FIGS. 7E-7G and 8A).

Furthermore, the NLRP3 dependent ASC nucleation-induced polymerization or oligomerization is considered as a unified mechanism of NLRP3 inflammasome activation (Lu et al., 2014, Cell 156:1193-1206; Yu et al., 2006, Cell. Death Differ. 13:236-249). It was observed that BHB prevented the ASC oligomerization in response to NLRP3 ligand ATP (FIG. 7H) and also prevented the ASC speck formation (FIG. 7I). Although not wishing to be bound by any particular theory, these results suggest that BHB's mechanism to block inflammasome activation is linked to its biophysical properties where it (a) controls an undetermined upstream event that reduces K⁺ efflux from macrophages in response to structurally diverse NLRP3 activators and (b) by inhibitory effects on ASC polymerization and speck formation suggesting direct effects on blocking the inflammasome assembly.

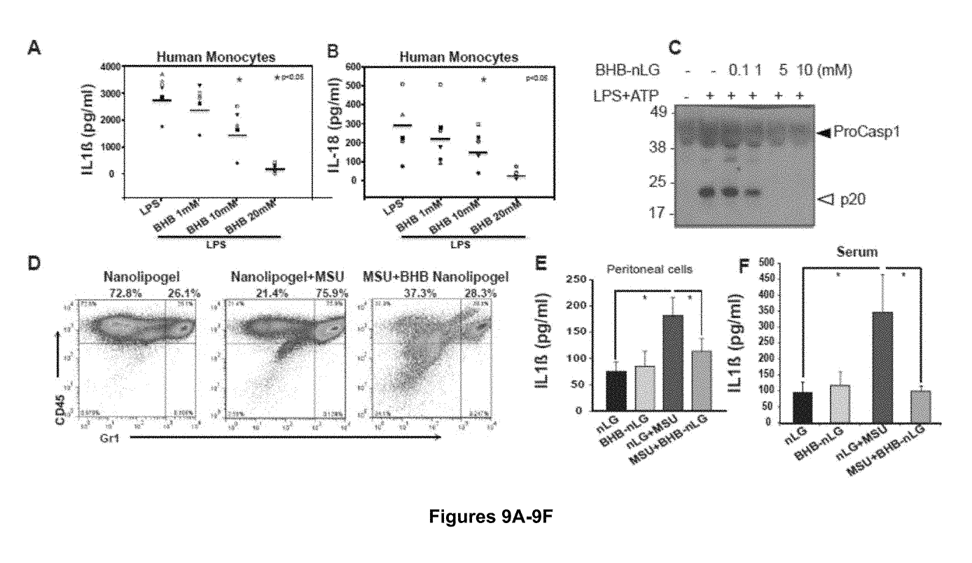

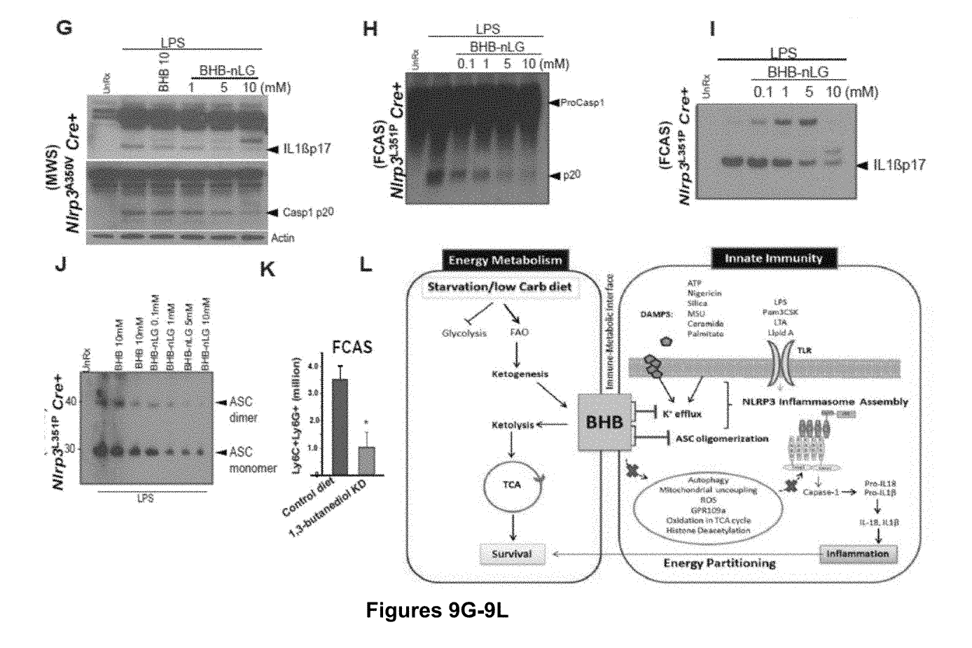

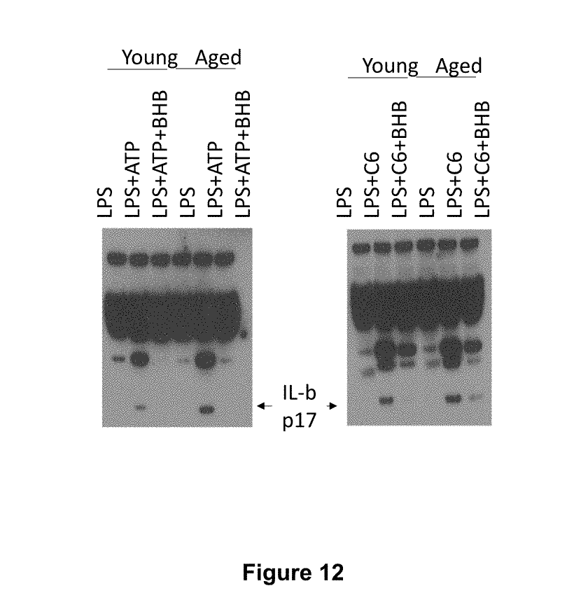

It was next examined whether delivery of BHB can inhibit the NLRP3 inflammasome in human monocytes and mouse models of NLRP3-driven inflammation in vivo. The BHB dose dependently inhibited IL-1β (FIG. 9A) and IL-18 (FIG. 9B) secretion in LPS stimulated monocytes without significantly affecting TNFα production (FIG. 8B). It has been observed that administration of BHB is insufficient to achieve sustained high serum concentration due to increased clearance (Newman and Verdin, 2014, Trends Endocrinol. Metab 25:42-52; Cotter et al., 2013, Am J Physiol Heart Circ Physiol. 304:H1060-1076; Lutas and Yellen, 2013, Trends Neurosci. 36:32-40). BHB with was complexed with nanolipogels (nLGs) in order to improve its bioavailability (Demento et al., 2011, Trends Biotechnol. 29:294-306). It was observed that BHB-nLGs were highly effective in inhibiting the NLRP3 inflammasome activation in macrophages (FIG. 9C). In order to activate the NLRP3 inflammasome, mice were injected with MSU intraperitoneally, and the influx of neutrophils and IL-1β levels were quantified after 4 hours using previously described methods (Martinon et al., Nature 440:237-241). The MSU driven inflammasome activation, resulting in increased neutrophil infiltration in the peritoneum, was inhibited in mice treated with BHB-nLGs (FIGS. 8C-8E and 9D) without directly impairing neutrophil migration (FIG. 8F) in contrast to mice given nLGs alone. Although not wishing to be bound by any particular theory, this result suggests direct effects in vivo of BHB-nLGs on NLRP3 driven neutrophil influx. In addition, compared to MSU challenged mice, the peritoneal cells derived from mice injected with BHB-nLGs produced less IL-1β (FIG. 9E) with a significant reduction in serum IL-1β levels (FIG. 9F), suggesting reduced inflammasome activation. BHB was also found to block IL-1β production after NLRP3 activation in neutrophils from young (3 month) and aged (24 month) mice (FIG. 12).

The therapeutic efficiency of BHB in knockin mice that mimic the human autoinflammatory diseases Muckle-Wells Syndrome (MWS) and Familial Cold Autoinflammatory Syndrome (FCAS) were examined. These knockin mice mimic these diseases due to gain of function mutation A350V and L351P in the NLRP3 which renders inflammasome constitutively active without the requirement of NLRP3 ligands (Brydges et al., 2009, Immunity 30:875-887). It was observed that the tamoxifen-induced Cre recombinase excision of floxed neomycin cassette in NLRP3^{L351P} activated the BMDMs by LPS alone (FIG. 10A). The treatment of BMDMs of MWS (FIGS. 8G and 9G) and FCAS (FIGS. 8G, 9H and 9I) mice with BHB-nLGs led to dose dependent inhibition of constitutive NLRP3 inflammasome activation. Importantly, when complexed with nLGs, the (D)-BHB inhibited inflammasome activation in FCAS macrophages with higher efficiency (FIGS. 10B and 10C). It was observed that in a mouse model of FCAS with constitutively active NLRP3 inflammasome, BHB directly prevented the ASC oligomerization in macrophages (FIGS. 8G and 9J).

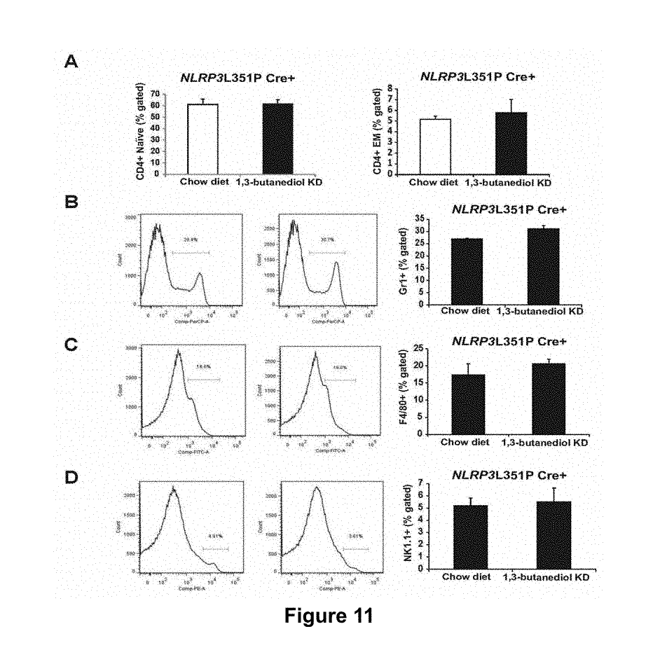

The NLRP3 mutation was activated by induction of Cre expression by tamoxifen treatment in adult mice at 6 weeks of age. Prior to tamoxifen injections, the Nlrp3^{L351P} Cre− and Nlrp3^{L351P} Cre+ mice were fed 1,3-butanediol ketone diesters (KD) for one week, which maintains BHB levels at fasting levels (0.75-1 mM). As reported previously, Nlrp3^{L351P} Cre+ mice develop severe neutrophila (Brydges et al., 2009, Immunity 30:875-887) in peritoneum within 3 days after induction of NLRP3 mutation. Compared to chow fed Nlrp3^{L351P} Cre+ control mice, the ketone diester treated FCAS mice exhibiting increased BHB levels were significantly protected from neutrophilia (FIG. 9L) and hyperglycemia (FIG. 10D) without altering the infiltration of CD11b⁺F4/80⁺ peritoneal macrophages (FIG. 10E). Furthermore, the ketone diester diet did not impact the overall frequency of T cells, macrophages or neutrophil numbers in spleen (FIG. 11). Although not wishing to be bound by any particular theory, these results suggest that because FCAS is caspase-1 dependent and downstream IL-1β and IL-18 do not control all the pathology (Brydges et al., 2009, Immunity 30:875-887), diets that elevate ketone body BHB may improve therapeutic outcome in patients by inhibiting the inflammasome.

The ability of fasting-induced metabolites such as BHB to inhibit the NLRP3 inflammasome without the need of surface GPR109a receptors or its oxidation for energetic purposes in macrophages signifies specificity and sophistication that avoids ‘bottleneck’ or competition for receptor occupancy and requirement of ATP generation. Thus, in states of extreme energy deficit such as during starvation, metabolic signals like BHB can operate to dampen innate immune responses, thus prioritizing resources to be allocated for essential functioning of ketone-dependent organs such as the brain and the heart (FIG. 9L).

### Example 2: BHB is Useful as a Treatment for Gout

Gout is induced by urate crystal accumulation that leads to inflammation in joints. Gout is characterized by neutrophil and macrophage infiltration which cause inflammation and tissue destruction in joints. Therapeutic strategies based on IL-1 inhibition are considered as an alternative treatment option, especially in patients with difficult-to-treat chronic gout. IL-1 can be regulated via multiple pathways that include NLRP3 inflammasome as well as inflammasome independent mechanisms especially in neutrophils. In gout, neutrophil influx is a major clinical sign that leads to inflammatory flares. Therefore, although not wishing to be bound by any particular theory, treatment strategies for gout that target both macrophage and neutrophils will have improved therapeutic outcome. The results described herein provide additional evidence that ketone metabolite beta-hydroxybutyrtae (BHB) suppresses the secretion of bioactive IL-1b (p17) from neutrophils. Given that gout is an age-dependent disease, the efficacy of BHB in controlling inflammation in aging neutrophils and in vivo in monosodium urate MSU induced inflammation was also examined. These data demonstrate that BHB is highly effective in suppressing neutrophil derived active IL-1b in vitro as well as in vivo. Thus, in one embodiment, BHB can be delivered in, within, and adjacent to a joint or joints of patients with gouty arthritis as a treatment for reducing inflammation and neutrophil influx.

The results of the experiments are now described.

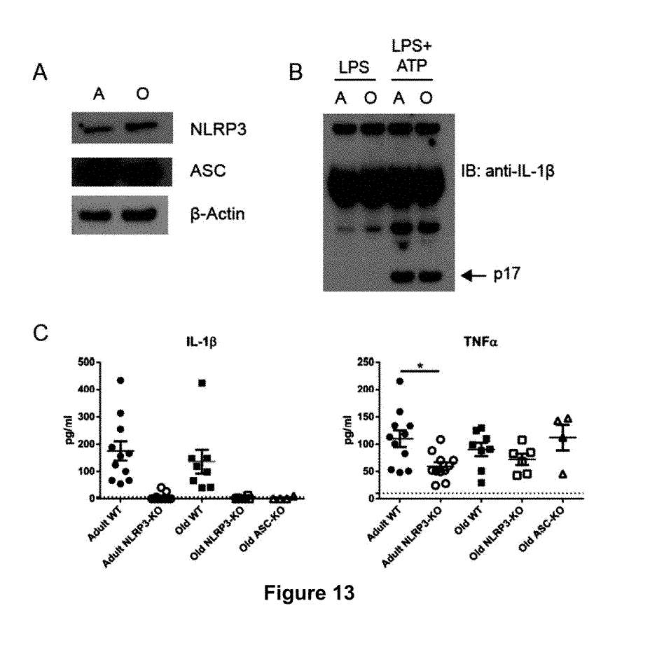

IL-1β secretion in adult and old neutrophils was found to be NLRP3-dependent (FIG. 13). Neutrophils from the femurs of adult and old mice were purified and analyzed for NLRP3 inflammasome components and activation. NLRP3, ASC, and β-Actin expression in unstimulated neutrophil cell lysates from adult and old mice were measured by Western blot (FIG. 13A). Supernatants from adult and old neutrophils stimulated with LPS±ATP were analyzed by Western blot for IL-β secretion (FIG. 13B). Neutrophils from adult and old mice of the indicated genotype were stimulated with LPS+ATP. IL-1β and TNFα were measured in the supernatants by Luminex (FIG. 13C). In FIGS. 13A and 13B, blots are representative of at least 3 independent experiments. Each adult and old sample is pooled from n=4-6 mice. In FIG. 13C, data is combined from two independent experiments. Each dot represents an individual mouse. *p<0.05. Statistical differences were calculated by 1-way ANOVA with Bonferroni's post test for multiple comparisons.

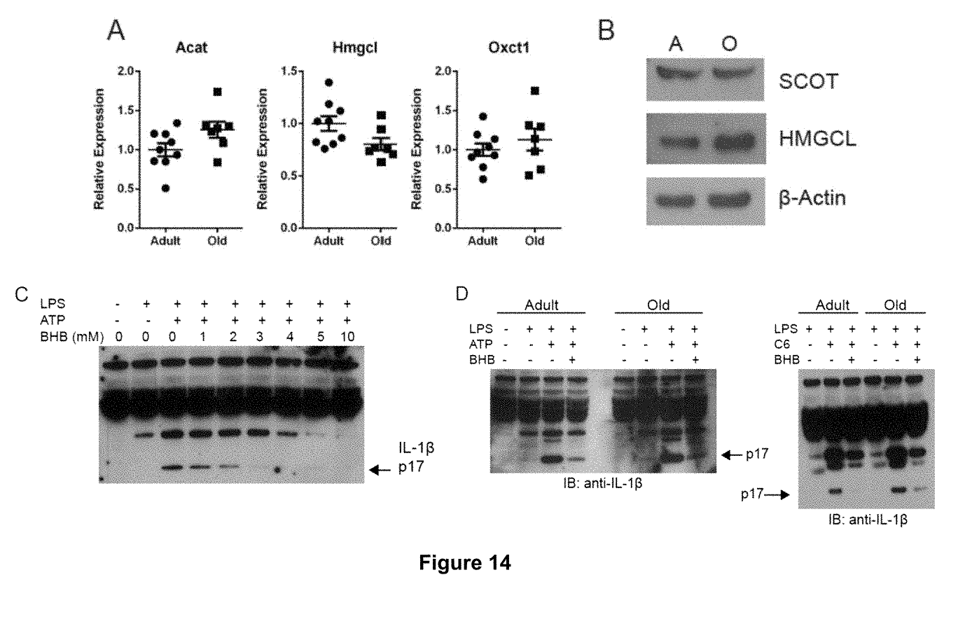

Neutrophils were found to contain ketone metabolism machinery and BHB was found to inhibit NLRP3 inflammasome activation (FIG. 14). Adult and old neutrophils were analyzed for ketogenic and ketolytic enzyme expression. Gene expression was measured by RT-PCR (FIG. 14A). Expression was normalized to Gapdh expression and was represented as expression relative to adult gene expression. SCOT, HMGCL, and β-Actin protein expression were assessed in unstimulated adult and old neutrophils by Western blot (FIG. 14B). Dose response to BHB was measured by assaying IL-1β secretion in supernatants from adult neutrophils (FIG. 14C). Western blots were generated of IL-1β in supernatants from adult and old neutrophils stimulated with LPS+ATP or LPS+ceramide (FIG. 14D). In FIG. 14A, each dot represents a pooled sample of n=2 mice. In FIGS. 14B-14D, each blot is representative of at least two independent experiments. Each sample analyzed by Western blot is pooled from n=4-6 mice per experiment.

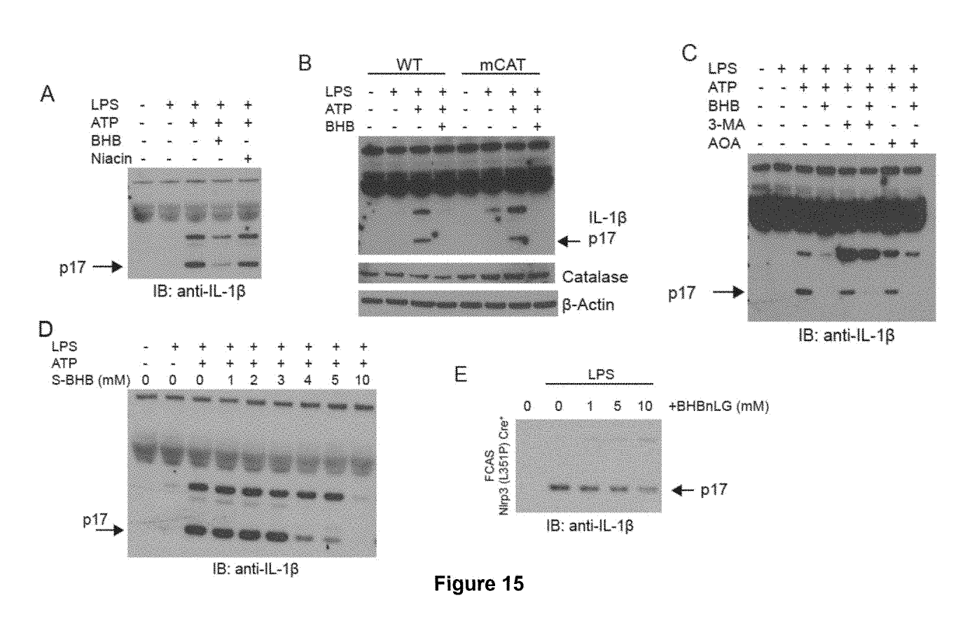

Inhibitory effects of BHB were observed to involve physically blocking inflammasome assembly (FIG. 15). Adult neutrophils were stimulated with LPS+ATP+BHB to test the mechanism by which BHB blocks IL-1β secretion. LPS-primed neutrophils were stimulated with ATP in the presence of BHB or niacin (FIG. 15A). Supernatants were analyzed for IL-1β secretion by Western blot. Wildtype of mCAT neutrophils were stimulated with LPS+ATP+BHB (FIG. 15B). Supernatants were analyzed for IL-1β secretion by Western blot. Catalase and β-Actin expression were measured by Western blot in neutrophil cell lysates. LPS-primed neutrophils were stimulated with ATP+BHB in the presence of 3-MA or AOA as indicated (FIG. 15C). Supernatants were analyzed for IL-1β secretion by Western blot. The enantiomer S-BHB was tested in a dose response for the ability to inhibit inflammasome activation (FIG. 15D). Supernatants were analyzed for IL-1β secretion by Western blot. FCAS neutrophils were incubated with 4-OHT followed by LPS priming. BHB nanolipogels were added to test inhibition of inflammasome activation (FIG. 15E). Supernatants were analyzed for IL-1β secretion by Western blot. For all blots, each sample is pooled from at least n=4 mice per experiment. Each blot is representative of at least two independent experiments.

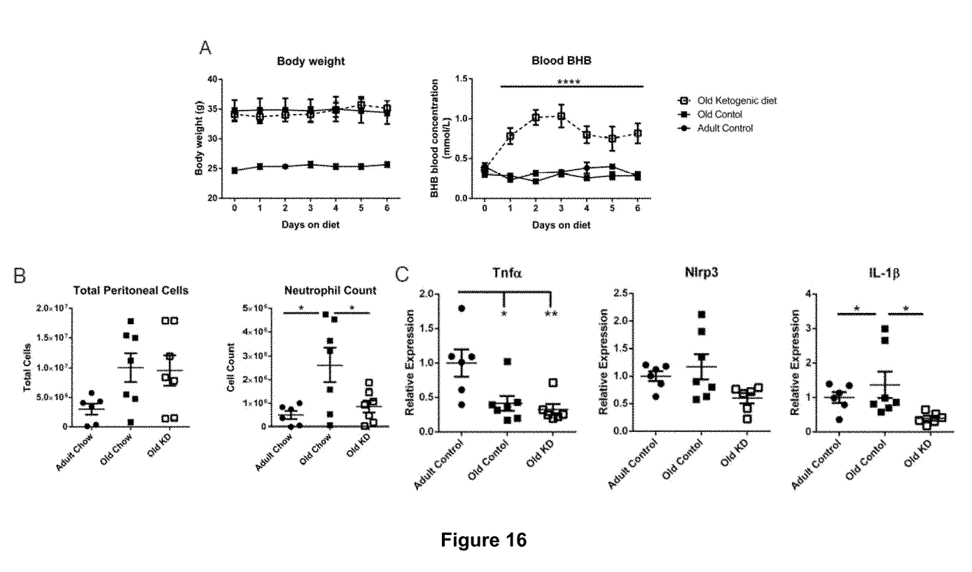

A ketogenic diet was observed to prevent neutrophil hyperactivation in a peritonitis model in old mice (FIG. 16). Old mice were fed a ketogenic diet for one week prior to MSU challenge to induce neutrophil infiltration and inflammasome activation (FIG. 16A). Body weights and blood BHB concentrations were measured daily. Four hours after MSU injection, total peritoneal cells were collected and analyzed by FACS to enumerate total neutrophil infiltration (FIG. 16B). Gene expression within peritoneal cells was measured by RT-PCR (FIG. 16C). Expression was normalized to Gapdh expression and data are represented as expression relative to adults. Data are pooled from two independent experiments (FIGS. 16B and 16C). Statistical differences were calculated by 2-way ANOVA (FIG. 16A) or 1-way ANOVA (FIG. 16B). Each dot represents an individual mouse. *p<0.05, ****p<0.0001.

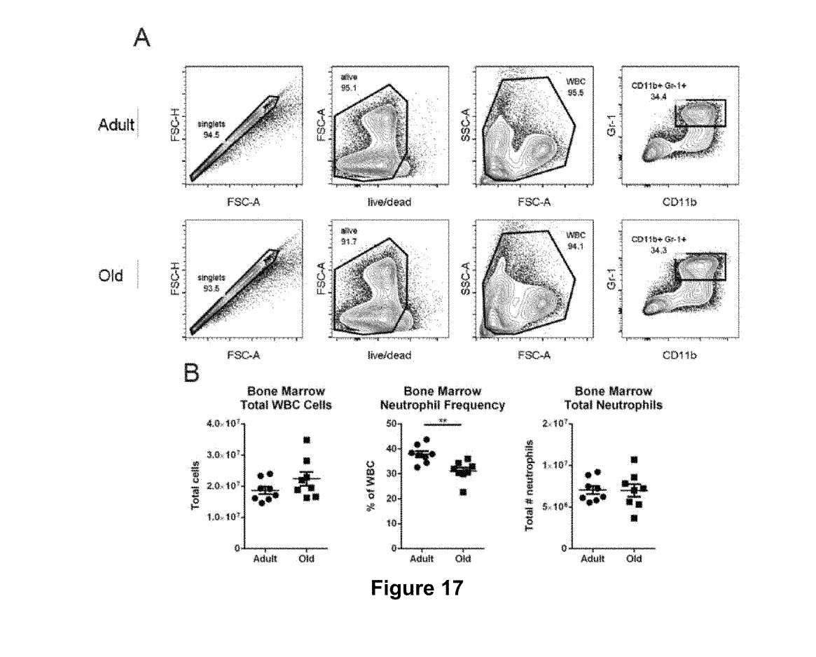

Neutrophil were further identified and enumerated (FIG. 17). Adult and old bone marrow was harvested from femurs and analyzed for neutrophils. A representative gating strategy was used to identify neutrophils from by multi-color flow cytometry (FIG. 17A). Enumeration of total cells after RBC lysis and calculation of total neutrophils (FIG. 17B). Data are representative of 2 independent experiments, each containing 8 mice per group. Statistical differences were calculated by unpaired student's t-test. Each dot represents an individual mouse. **p<0.01.

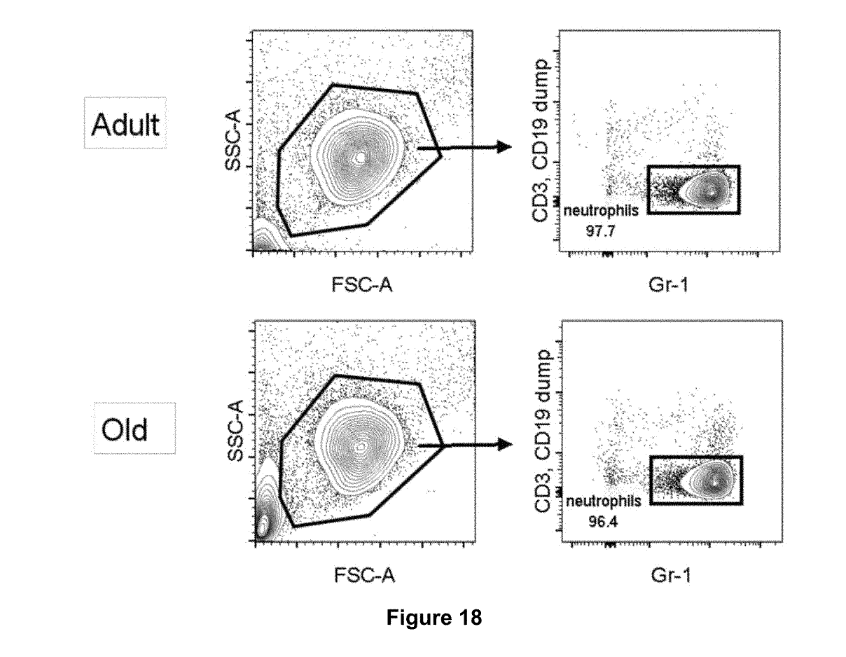

The purity of adult and old neutrophils was assessed after magnetic enrichment (FIG. 18). Adult and old neutrophils were enriched from bone marrow for all ex vivo stimulation experiments. Representative flow cytometry analysis showing comparable purity between adult and old samples (FIG. 18).

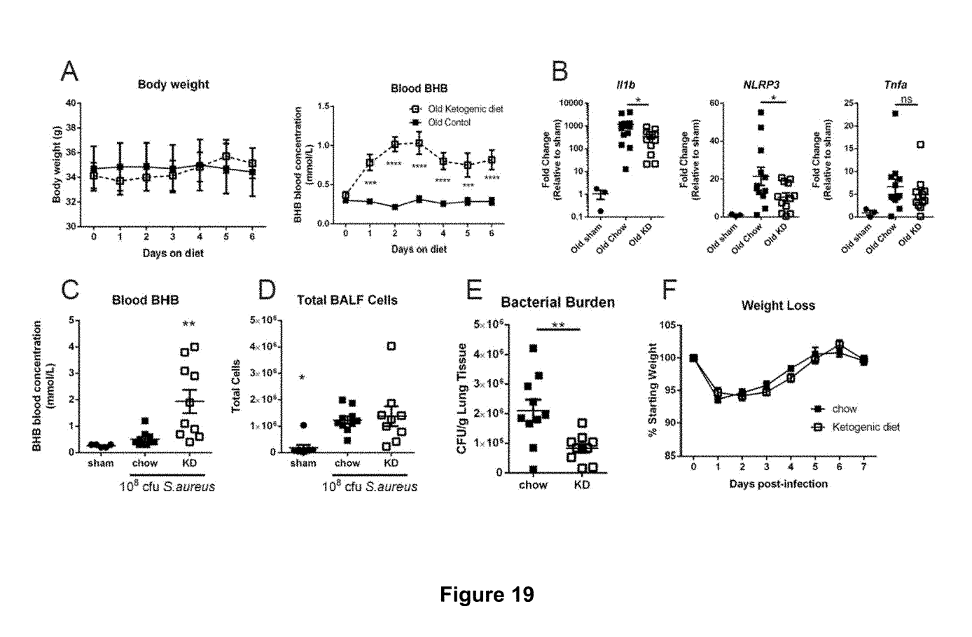

It was also observed that BHB did not increase infection severity but still prevented neutrophil hyperactivation during peritonitis. Mice were fed a ketogenic diet for 1 week prior to an increase in BHB levels and then infection or peritonitis was induced by injection of monosodium urate. Body weights and blood BHB concentrations were measured daily in old mice during ketogenic diet feeding, prior to MSU injection (FIG. 19A). Blood BHB levels in old mice fed ketogenic diet were observed. Old mice fed chow and ketogenic diet were treated with i.p urate injections to induce periitonitis. The peritoneal cells were isolated and pro-inflammatory cytokine Il1b, Nlrp3 and Tnf gene expression was measured by RT-PCR (FIG. 19B). Expression was normalized to Gapdh expression and data are represented as expression relative to sham Old mice. Blood BHB levels were measured in adult mice 24 hr post-infection with Staphylococcus aureus (FIG. 19C). Total cells collected from brocho-aleveolar lavage BAL fluid from lungs 24 hr post-infection were quantified (FIG. 19D). Bacterial burdens in lung tissue were determined 24 hr after infection (FIG. 19E). Body weights were measured daily for 7 days following S. aureus infection until all mice returned to baseline body weight. (FIG. 19F). All data are pooled from at least two independent experiments. Statistical differences were calculated by ANOVA or unpaired student's t-test. Each dot represents an individual mouse. *p<0.05, ****p<0.0001.

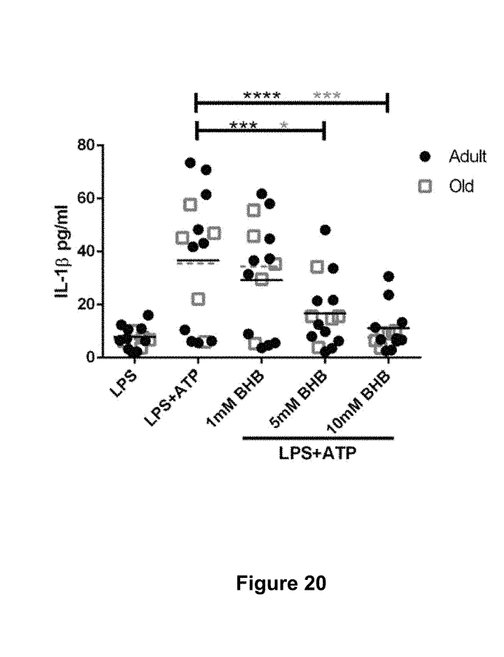

BHB was also observed to inhibit IL1B production from human neutrophils irrespective of age. Peripheral blood neutrophils from adult (30-40 years) and old (65-75 years) were enriched and stimulated as indicated (FIG. 20). IL-1β secretion was measured in culture supernatants by ELISA. Data are expressed as mean±S.E.M (*p<0.01).

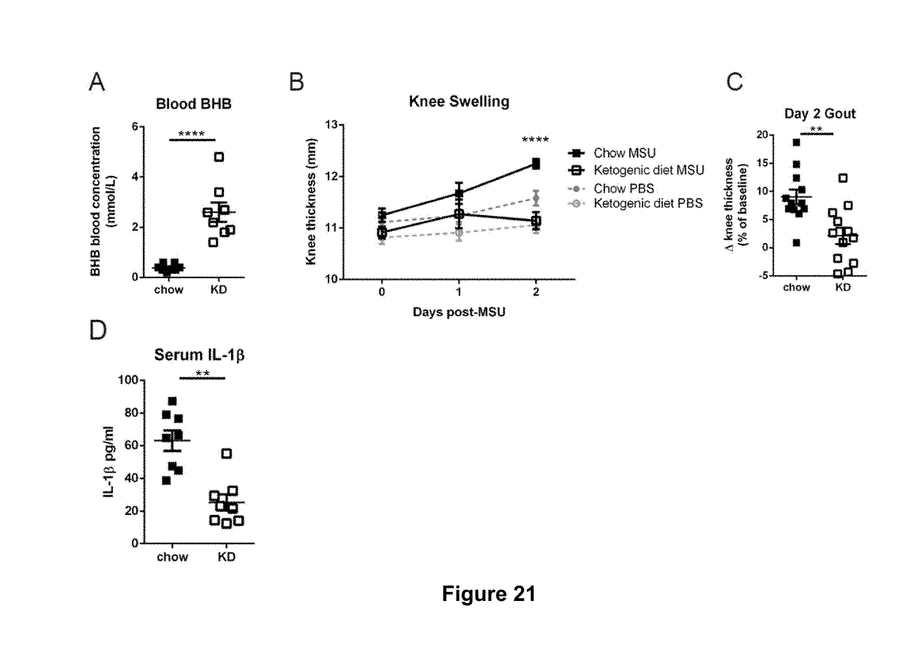

Increasing the levels of BHB was found to protect against Gout-induced inflammation in rats. Rats were fed a ketogenic diet for 1 week prior to the induction of gout by intra-articular injection of MSU. Blood BHB levels were measured in rats after 1 week ketogenic diet feeding, prior to injection with MSU (FIG. 21A). Knee thickness was measured daily (FIG. 21B). The increase in knee swelling relative to baseline thickness was measured 2 days after gout induction (FIG. 21C). Serum IL-1β was measured by ELISA on day 2 post-MSU injection (FIG. 21D).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating or preventing an NLRP3 inflammasome-related disease or disorder, the method comprising administering a therapeutically effective amount of a composition comprising at least one NLRP3 inflammasome inhibitor to a subject in need thereof.

2.-3. (canceled)

4. The method of claim 1, wherein the NLRP3 inflammasome inhibitor is selected from the group consisting of β-hydroxybutyrate (BHB), γ-hydroxybutyrate (GHB), α-hydroxybutyrate (α-HB), polyhydroxybutyrate, a salt thereof, and any combinations thereof.

5. (canceled)

6. The method of claim 1, wherein the NLRP3 inflammasome inhibitor is (S)-β-hydroxybutyrate [(S)-BHB].

7. The method of claim 1, wherein the NLRP3 inflammasome inhibitor is conjugated to a nanoparticle.

8. The method of claim 7, wherein the nanoparticle is a nanolipogel comprising at least one liposome and a core.

9. (canceled)

10. The method of claim 8, wherein the liposome is comprised of cholesterol, at least one phosphatidylcholine lipid, and at least one PEG-lipid.

11-12. (canceled)

13. The method of claim 10, wherein the molar ratio of cholesterol to photphatidylcholine lipid to PEG-lipid is about 2:1:0.1

14. The method of claim 8, wherein the core comprises the at least one inhibitor, at least one host material, and at least one photoinitiator, wherein the host material is at least one cyclodextrin.

15-18. (canceled)

19. The method of claim 1, wherein the disease or disorder is selected from the group consisting of gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multisystem inflammatory disease (NOMID); Muckle-Wells Syndrome (MWS) and Familial Cold Autoinflammatory syndrome (FCAS).

20. The method of claim 1, further comprising administering to the subject a ketogenic diet.

21. (canceled)

22. A composition comprising at least one NLRP3 inflammasome inhibitor conjugated to a nanoparticle.

23. The composition of claim 22, wherein the nanoparticle is a nanolipogel comprising at least one liposome and a core.

24. (canceled)

25. The composition of claim 22, wherein the liposome comprises cholesterol, at least one phosphatidylcholine lipid, and at least one PEG-lipid.

26-27. (canceled)

28. The composition of claim 25, wherein the molar ratio of cholesterol to photphatidylcholine lipid to PEG-lipid is about 2:1:0.1

29. The composition of claim 23, wherein the core is comprised of the at least one inhibitor, at least one host material, and at least one photoinitiator, wherein the host material is at least one cyclodextrin.

30.-33. (canceled)

34. The composition of claim 22, further comprising at least one pharmaceutically acceptable carrier.

35. A method for treating or preventing an NLRP3 inflammasome-related disease or disorder, the method comprising administering a therapeutically effective amount of a composition comprising at least one NLRP3 inflammasome inhibitor to a joint in a subject in need thereof.

36.-37. (canceled)

38. The method of claim 35, wherein the NLRP3 inflammasome inhibitor is selected from the group consisting of β-hydroxybutyrate (BHB), γ-hydroxybutyrate (GHB), α-hydroxybutyrate (α-HB), polyhydroxybutyrate, a salt thereof, and any combinations thereof.

39. (canceled)

40. The method of claim 35, wherein the NLRP3 inflammasome inhibitor is (S)-β-hydroxybutyrate [(S)-BHB].

41. The method of claim 35, wherein the disease or disorder is selected from the group consisting of gout, arthritis, atherosclerosis, type-2 diabetes, diabetic nephropathy, glomerulonephritis, acute lung injury (ALI), thymic degeneration, steatohepatitis, Alzheimer's disease, multiple sclerosis, silicosis, age-related bone loss, age-related functional decline, Macular degeneration, neonatal-onset multi system inflammatory disease (NOMID), Muckle-Wells Syndrome (MWS) and Familial Cold Autoinflammatory syndrome (FCAS).

42. The method of claim 35, wherein the composition is administered by injection directly into the joint or topically on or around the joint.

43. (canceled)
